(19) United States
(12) Patent Application Publication
(10) Pub. No.: US 2003/0152956 A1
(43) Pub. Date: Aug. 14, 2003

(54) Method of examining allergic disease

(76) Inventors: Noriko Ohtani, Kawasaki-shi, Kanagawa (JP); Keiko Matsui, Kawasaki-shi, Kanagawa (JP); Nei Yoshida, Kawasaki-shi, Kanagawa (JP); Yuji Sugita, Kawasaki-shi, Kanagawa (JP); Kenji Izuhara, Saga-shi, Saga (JP)

Correspondence Address: LAHIVE & COCKFIELD, 28 STATE STREET, BOSTON, MA, 02109, US

(21) Appl. No.: 10/204,752

(22) PCT Filed: Dec. 21, 2001
(86) PCT No.: PCT/JP01/11287

(30) Foreign Application Priority Data
Dec. 26, 2000 (JP) 2000-396166

(51) Int. Cl. C12Q 1/68; G01N 33/53

(52) U.S. Cl. 435/6; 435/7.1

(57) ABSTRACT

Six genes, whose expressions were greatly changed in a plurality of cells by stimulating respiratory tract epithelial cells with IL-4 or IL-13, were obtained as allergy related genes. This invention provides a method of testing for allergic diseases, and method of screening for compounds useful in treating such diseases, that use as indicators, expression levels of these genes in biological samples.

### TECHNICAL FIELD

The present invention relates to a method of testing for an allergic disease.

### BACKGROUND ART

Bronchial asthma is considered to be a multifactorial disease. In other words, bronchial asthma is caused by the interaction of many different genes, each of which is influenced by various environmental factors. Thus, it has been extremely difficult to identify a specific gene which causes bronchial asthma.

Currently, bronchial asthma is categorized as a chronic inflammatory disease of the respiratory tract. It has been pointed out that allergic reactions at the respiratory tract mucosa and bronchial smooth muscle is closely involved in pathologic formation of bronchial asthma. Therefore, understanding the condition of allergic reactions in these tissues is an important issue in diagnosis of bronchial asthma. In addition, control of allergic reactions is an issue in treatment of bronchial asthma.

On the other hand, the expression of mutated or defective genes, or overexpression or reduction of the expression of specific gene is thought to be involved in allergic diseases. To elucidate the role of gene expression in diseases, it is necessary to understand how a gene is involved in triggering disease onset and how expression of the gene is altered by external stimulants such as drugs.

Incidentally, atopic diathesis that is accompanied by hyperproduction of IgE antibodies is seen in many bronchial asthma patients. Many causes are considered for bronchial asthma, but there is no doubt that atopic diathesis is a cause of hypersensitivity in many patients. It has been predicted that the mechanism of respiratory tract occlusion in asthmatic attack is contraction of the bronchial smooth muscle, or edema and respiratory tract endocrine enhancement of the respiratory tract mucosa. I-type allergic reaction in the respiratory tract due to exposure to pathogenic allergen has an important role in such changes in the respiratory tract.

In recent years, IL-4 and IL-13 have been suggested to have important roles in the onset of bronchial asthma. Therefore, for example, in the respiratory tract epithelial cells and bronchial smooth muscles, genes that change their expression level due to IL-4 and IL-13 are thought to be related to bronchial asthma. However, based on such concept, there have been no reports on isolation of genes that specifically change their expression level due to IL-4 and IL-13.

In recent diagnosis of allergic diseases, history taking, and confirmation of the patient's family history and own anamnesis are important factors in general. In addition, for diagnosis of allergy based on more objective information, a test method using patient's blood sample and method for observing patient's immune response to allergen are also performed. Examples of the former method are the allergen-specific IgE measurement, leukocyte histamine release test, lymphocyte stimulating test, or the like. Presence of an allergen-specific IgE is a proof for the allergic reaction to that allergen. However, in some patients, allergen-specific IgE may not necessarily be detected. Furthermore, the assay principle of IgE requires performing tests for all of the allergens necessary for diagnosis. Leukocyte histamine release test and lymphocyte stimulating test are the methods for observing the immune system reaction toward a specific allergen in vitro. These methods are complicate in operation.

On the other hand, another method is also known, wherein the immune response observed when a patient is actually contacted with an allergen is used for diagnosing an allergy (latter method). Such a test includes the prick test, scratch test, patch test, intradermal reaction, or induction test. Indeed these tests allow the direct diagnosis of patient's allergic reaction but they can be said to be highly invasive tests wherein patients are actually exposed to allergen.

In addition, regardless of the allergen types, test methods for proving the involvement of allergic reaction are also attempted. For example, a high serum IgE titer may indicate the occurrence of allergic reaction in the patient. The serum IgE titer is information corresponding to the total amount of allergen-specific IgE. Though it is easy to determine the total amount of IgE regardless of the type of allergen, IgE titer may be reduced in some patients with a non-atopic bronchitis or the like.

Therefore, a marker (indicator) for an allergic disease that is not only less risky to patients but also capable of readily providing information necessary for diagnosis would be useful. Since such markers are thought to be profoundly involved in triggering disease onset, they may become the important target in not only diagnosis but also control of allergic symptoms.

### DISCLOSURE OF THE INVENTION

An objective of the present invention is to provide an indicator enabling the test for allergic disease, in particular. Another objective of the invention is to provide a method of testing for an allergic disease and a method of screening for a candidate compound for a therapeutic agent for an allergic disease based on the indicator.

Deep involvement of IL-4 and IL-13 in allergic reaction has been suggested by several reports. For example, in an IL-4 knockout mouse (Yssel, H and Groux, H: Int. Arch. Allergy Immunol., 121; 10-18, 2000) and in a STAT6 knockout mouse (Akimoto, T. et al.: J. Exp. Med., 187, 1537-1542, 1998), respiratory tract hypersensitivity disappears. In a mouse model, IL-13 is involved in forming asthma-like pathology regardless of IgE production and Th2 type (Wills-Karp, M. et al.: Science, 282, 2258-2261, 1998; Grunig, G. et al.: Science, 282, 2261-2263, 1998; Zhu, Z. et al.: J. Clin. Invest., 103, 779-788, 1999).

Additionally, IL-4 receptors and IL-13 receptors are highly expressed in human respiratory tract epithelial cells and bronchial smooth muscles (Heinzmann, A. et al.: Hum. Mol. Genet., 9: 549-559, 2000). Accordingly, these tissues are thought to be target cells of IL-4 and IL-13. On the other hand, SNP present in IL-4 receptor α and IL-13 were shown to be one of the genetic causes of allergic diseases (Mitsuyasu, H., et al.: Nature Genet., 19, 119-120, 1998; Mitsuyasu, H., et al.: J. Immunol., 162: 1227-1231, 1999; Kruse, S., et al.: Immnol., 96, 365-371, 1999; Heinzmann, A. et al.: Hum. Mol. Genet., 9: 549-559, 2000). Furthermore, inhibition of IL-4 or IL-13 function by soluble IL-4 receptor □ was shown to be effective as treatment for bronchial asthma (Borish, L. C. et al.: Am. J. Respir. Crit. Care Med., 160: 912-922, 1999).

According to the above, a strong relationship to allergic reactions, mainly respiratory symptoms in particular, has been suggested for IL-4 and IL-13. That is, genes constituting signal transduction pathway due to IL-4 and IL-13 may be genes that are closely related to allergic reactions. Therefore, by isolating these genes and elucidating their relation to allergic reactions, novel targets for treatment of allergic diseases can be found.

Based on such line of thought, the inventors thought that, if genes indicating changes in expression levels were searched for when human bronchial epithelial cells were treated with IL-4 and IL-13, genes relating to allergic reactions could be isolated. There is a report that attempted, using a similar approach, to isolate genes whose expression level changed by IL-4 and IL-13 treatment (Wang et al., Immunology 2000, Seattle, May 12-16, 2000). However, since in known searching methods, the number of lots of cells used for analysis is small, and the range of changes in expression levels are not clear, specificity towards stimulation from IL-4 and IL-13 cannot be expected.

Therefore, to isolate genes that respond with higher specificity towards IL-4 and IL-13 stimulation, the inventors increased the number of lots of cells that become the object of analysis, and furthermore, selected those in which the change in expression level reached twice as much or more. Next, in the respiratory tract epithelial cells stimulated with IL-4 and IL-13, significant elevation of expression level of genes selected this way was confirmed. Based on the findings mentioned above, the inventors succeeded in elucidating the existence of the following 6 genes, which have close relationship to allergic diseases.

carboxypeptidase M

cathepsin C

endothelin-A receptor

osteoblast specific factor 2 (OSF2os)

DD96(MAP17)

and dioxin-inducible cytochrome P450 (CYP1B1)

Based on the findings mentioned above, the inventors found that tests for allergic diseases become possible by using these genes and proteins encoded by these genes as indicators, and completed this invention. Additionally, the inventors found that screening of therapeutic agents for allergic diseases is possible by using expression levels of these genes or activities of proteins encoded by these genes as indicators, and completed this invention.

That is, this invention relates to the following testing method and screening methods.

[1] A method of testing for an allergic disease, the method comprising the steps of:

a) measuring the expression level of one or more genes selected from the group consisting of carboxypeptidase M, cathepsin C, endothelin-A receptor, osteoblast specific factor 2, DD96, and CYP1B1 in a biological sample from a subject, and

b) comparing the expression level measured in (a) with that in a biological sample from a normal healthy subject.

[2] The method of [1], wherein the allergic disease is bronchial asthma.

[3] The method of [1], wherein the expression level is measured by PCR of the cDNA for the one or more genes.

[4] The method of [1], wherein the expression level is measured by detecting the protein encoded by the one or more genes.

[5] A reagent for testing for an allergic disease, said reagent comprising an oligonucleotide that is at least 15 nucleotides long and that has a nucleotide sequence complementary to a polynucleotide having a nucleotide sequence of one or more genes selected from the group consisting of carboxypeptidase M, cathepsin C, endothelin-A receptor, osteoblast specific factor 2, DD96(MAP17), and CYP1B1or to a complementary strand of the polynucleotide.

[6] A reagent for testing for an allergic disease, said reagent comprising an antibody that recognizes a peptide having an amino acid sequence of one or more proteins selected from the group consisting of carboxypeptidase M, cathepsin C, endothelin-A receptor, osteoblast specific factor 2, DD96(MAP17), and CYP1B1.

[7] A method of screening for a therapeutic agent for an allergic disease, the method comprising the steps of:

(1) contacting a candidate compound with a cell that expresses one or more genes selected from the group consisting of carboxypeptidase M, cathepsin C, endothelin-A receptor, osteoblast specific factor 2, DD96(MAP17), and CYP1B1, and/or one or more genes functionally equivalent thereto,

(2) measuring the expression level of the one or more genes, and

(3) selecting a compound that lowers the expression level, compared to a control.

[8] The method of [7], wherein the cell is a respiratory tract epithelial cell line.

[9] A method of screening for a therapeutic agent for an allergic disease, the method comprising the steps of:

(1) administering a candidate compound to a test animal,

(2) measuring, in a biological sample from the test animal, the expression level of one or more genes selected from the group consisting of carboxypeptidase M, cathepsin C, endothelin-A receptor, osteoblast specific factor 2, DD96(MAP17), and CYP1B1, and/or one or more genes functionally equivalent thereto, and

(3) selecting a compound that lowers the expression level of the one or more genes, compared to a control.

[10] A method of screening for a therapeutic agent for an allergic disease, the method comprising the steps of:

(1) contacting a candidate substance with a cell transfected with a vector having a transcription regulatory region of one or more genes selected from the group consisting of carboxypeptidase M, cathepsin C, endothelin-A receptor, osteoblast specific factor 2, DD96(MAP17), and CYP1B1, and/or one or more genes functionally equivalent thereto, and a reporter gene that is expressed under the control of the transcription regulatory region,

(2) measuring activity of the reporter gene, and

(3) selecting a compound that lowers the expression level of the reporter gene, compared to a control.

[11] A method of screening for a therapeutic agent for an allergic disease, the method comprising the steps of:

(1) contacting a candidate substance with one or more proteins selected from the group consisting of carboxypeptidase M, cathepsin C, endothelin-A receptor, osteoblast specific factor 2, DD96(MAP17), and CYP1B1, and/or one or more proteins functionally equivalent thereto,

(2) measuring activity of the one or more proteins, and

(3) selecting a compound that lowers the activity, compared to a control.

[12] A therapeutic agent for an allergic disease, said agent comprising, as an active ingredient, a compound obtained by the method of any one of [7], [9], [10], and [11].

[13] A therapeutic agent for an allergic disease, said agent comprising, as a major component, an antisense DNA against one or more genes selected from the group consisting of carboxypeptidase M, cathepsin C, endothelin-A receptor, osteoblast specific factor 2, DD96(MAP17), and CYP1B1 or against a portion thereof.

[14] A therapeutic agent for an allergic disease, said agent comprising, as a major component, an antibody that binds to one or more proteins selected from the group consisting of carboxypeptidase M, cathepsin C, endothelin-A receptor, osteoblast specific factor 2, DD96, and CYP1B1.

[15] Use of a transgenic non-human vertebrate in which the expression level of one or more genes selected from the group consisting of carboxypeptidase M, cathepsin C, endothelin-A receptor, osteoblast specific factor 2, DD96(MAP17), and CYP1B1, and/or one or more genes functionally equivalent thereto is elevated in respiratory tract epithelial cells, as an animal model for an allergic disease.

[16] A kit for screening for a candidate compound for a therapeutic agent for an allergic disease, the kit comprising

a polynucleotide that is at least 15 nucleotides long and that hybridizes to a nucleotide sequence of one or more genes selected from the group consisting of carboxypeptidase M, cathepsin C, endothelin-A receptor, osteoblast specific factor 2, DD96(MAP17), and CYP1B1 or to a complementary sequence thereof, and

a cell that expresses a gene comprising a nucleotide sequence of one or more genes selected from the group consisting of carboxypeptidase M, cathepsin C, endothelin-A receptor, osteoblast specific factor 2, DD96(MAP17), and CYP1B1.

[17] A kit for screening for a candidate compound for a therapeutic agent for an allergic disease, the kit comprising

an antibody that recognizes a peptide having an amino acid sequence of one or more proteins selected from the group consisting of carboxypeptidase M, cathepsin C, endothelin-A receptor, osteoblast specific factor 2, DD96(MAP17), and CYP1B1 , and

a cell that expresses a gene comprising a nucleotide sequence of one or more genes selected from the group consisting of carboxypeptidase M, cathepsin C, endothelin-A receptor, osteoblast specific factor 2, DD96(MAP17), and CYP1B1.

Otherwise, this invention relates to a method of treating an allergic disease, the method comprising the step of administering a compound that can be obtained by the method of any one of [7], [9], [10], and [11]. Furthermore, this invention relates to use of a compound that can be obtained by the method of any one of [7], [9], [10], and [11], for producing a pharmaceutical composition for treating an allergic disease. Additionally, this invention relates to a method of treating an allergic disease, the method including the step of administering an antisense DNA against the aforementioned gene or an antibody that binds to the aforementioned protein. Also, this invention relates to a use of an antisense DNA against the aforementioned gene or an antibody that binds to the aforementioned protein, for producing a pharmaceutical composition for treating an allergic disease.

In addition, the existence of all of these 6 genes has been elucidated. The nucleotide sequences of the 6 genes and the amino acid sequences encoded by them are shown in the following SEQ ID NOs.

| | Nucleotide sequence | Amino acid sequence |
|---|---|---|
| Carboxypeptidase M | SEQ ID NO: 26 | SEQ ID NO: 27 |
| Cathepsin C | SEQ ID NO: 28 | SEQ ID NO: 29 |
| Endothelin-A receptor | SEQ ID NO: 30 | SEQ ID NO: 31 |
| Osteoblast specific factor 2 | SEQ ID NO: 32 | SEQ ID NO: 33 |
| DD96(MAP17) | SEQ ID NO: 34 | SEQ ID NO: 35 |
| CYP1B1 | SEQ ID NO: 36 | SEQ ID NO: 37 |

The already elucidated utility of the 6 genes used in this invention are shown below. For all of the genes, as indicated below, it was not known that their expression is enhanced in respiratory tract epithelial cells in response to IL-4 and IL-13.

Carboxypeptidase M:

Regarding carboxypeptidase M, there is no report in particular regarding its utility.

Cathepsin C:

A homologue of cathepsin C is shown to be useful as a diagnostic agent or a therapeutic agent for a monocytic and a macrophagic disease. Also, its use as a therapeutic agent or a diagnostic agent for type II collagen has been reported.

Endothelin-A Receptor:

It has been shown that this can be used for measuring endothelin, and for screening for antagonists against endothelin. Otherwise, it has been reported that this can be used as an inhibitor for an endothelin related disease.

Osteoblast Specific Factor 2:

Its use as a diagnostic agent for bone metabolism, or as a tumor marker has been reported. Furthermore, regarding DNA and monoclonal antibodies, their use as therapeutic agents for thoracic, large intestine, or gastrointestinal cancer have been indicated. Also, use as an inhibitor, diagnostic agent, or therapeutic agent for heart, lung, and inflammatory diseases have been known.

DD96(MAP17):

It has been reported that 5′-EST of DD96(MAP17) may be used for diagnosis, forensic medicine, gene therapy, and chromosome mapping.

CYP1B1:

It has been shown that this can be used as a diagnostic marker for renal cancer and glaucoma.

In the present invention, allergic disease is a general term for diseases in which allergic reactions is involved. More specifically, for a disease to be considered allergic, the allergen must be identified, a strong correlation between exposure to the allergen and the onset of the pathological change must be demonstrated, and the pathological change has been proven to have an immunological mechanism. Herein, an immunological mechanism means that the leukocytes show an immune response to allergen stimulation. Examples of allergens are the mite antigen, pollen antigen, etc.

Representative allergic diseases are bronchial asthma, allergic rhinitis, pollinosis, insect allergy, etc. Allergic diathesis is a genetic factor that is inherited from allergic parents to children. Familial allergic diseases are also called atopic diseases, and their causative factor that can be inherited is atopic diathesis. Among atopic diseases, asthma is a general term for diseases accompanied with respiratory organ symptoms.

The method of testing for an allergic disease of this invention includes the steps of measuring the expression level of one or more genes selected from the group consisting of carboxypeptidase M, cathepsin C, endothelin-A receptor, osteoblast specific factor 2 (OSF2os), DD96(MAP17), and CYP1B1 in a biological sample from a subject, and comparing the measured value with that for a normal healthy subject. As a result of comparing the two, if expression is enhanced compared to that of a normal healthy subject, the subject is judged to have an allergic disease. In this invention, the six aforementioned genes that can be indicators of allergic diseases are called indicator genes. Unless otherwise stated, the term “indicator gene” as used herein means any one or more genes selected from the six genes mentioned above.

In the method of testing for allergies based on this invention, indicator genes that are the target of expression level and activity measurements are selected from the six aforementioned indicator genes. Therefore, testing based on this invention can be performed by measuring the expression level and activity of any one of the genes of the 6 types of genes. Furthermore, in this invention, accuracy of the testing can be enhanced by measuring a plurality of these genes in combination. Since bronchial asthma patients form a heterogeneous group, more accurate diagnosis can be performed using a plurality of genes as indicators. Specifically, at least one, preferably 2 or more, more preferably 3 or more, and even more preferably 4 or 5 types or more indicator genes can be combined.

In this invention, expression levels of indicator genes include transcription of these genes to mRNA, and translation into proteins. Therefore, the method of testing for an allergic disease of this invention is performed based on comparing the strength of expression of mRNA corresponding to the aforementioned genes, or the expression level of proteins encoded by the aforementioned genes.

The measurement of expression level of indicator genes in the testing of allergic diseases of this invention can be carried out according to known genetic analysis methods. Specifically, one can use, for example, a hybridization technique that uses nucleic acids that hybridize to these genes as probes, or a gene amplification technique that uses DNAs that hybridize to the genes used in this invention as primers can be used.

The probes or primers used for the testing of this invention can be set based on the nucleotide sequences of the aforementioned indicator genes. The nucleotide sequences of the aforementioned indicator genes are well known. The GenBank accession numbers for the nucleotide sequence of each of the indicator genes are indicated in the Examples.

Genes of higher animals are generally accompanied by polymorphism in a high frequency. There exist many molecules that produce isoforms comprising different amino acid sequences from each other during the splicing process. Any genes associated with allergy which have a similar activity to that of the indicator gene are included in the indicator gene of the present invention, even though they carry mutation in the nucleotide sequence due to polymorphism and isoform.

As a primer or probe can be used a polynucleotide comprising the nucleotide sequence of the indicator gene or at least 15 nucleotides that are complementary to the complementary strand thereof. Herein, the term “complementary strand” means one strand of a double stranded DNA composed of A:T (U for RNA) and G:C base pairs to the other strand. In addition, “complementary” means not only those completely complementary to a region of at least 15 continuous nucleotides, but also having a homology of at least 70%, preferably at least 80%, more preferably 90%, and even more preferably 95% or higher. The degree of homology between nucleotide sequences can be determined by the algorithm, BLAST, etc.

Such polynucleotides are useful as the probe to detect an indicator gene, or as the primer to amplify the indicator gene. When used as a primer, those polynucleotides comprises usually 15 bp˜100 bp, preferably 15 bp˜35 bp of nucleotides. When used as a probe, DNAs comprising the whole sequence of the indicator gene (or a complementary strand thereof), or a partial sequence thereof that contains at least 15-bp nucleotides. When used as a primer, the 3′ region thereof must be complementary to the indicator gene, while the 5′ region can be linked to a restriction enzyme-recognition sequence or tag.

“Polynucleotides” in the present invention may be either DNA or RNA. These polynucleotides may be either synthetic or naturally-occurring. Also, DNA used as a probe for hybridization is usually labeled. Examples of labeling methods are those as described below. Herein, the term “oligonucleotide” means a polynucleotide with relatively low degree of polymerization. Oligonucleotides are included in polynucleotides. The labeling methods are as follows:

nick translation labeling using DNA polymerase I;

end labeling using polynucleotide kinase;

fill-in end labeling using Klenow fragment (Berger, S L, Kimmel, A R. (1987) Guide to Molecular Cloning Techniques, Method in Enzymology, Academic Press; Hames, B D, Higgins, S J (1985) Genes Probes: A Practical Approach. IRL Press; Sambrook, J, Fritsch, E F, Maniatis, T. (1989) Molecular Cloning: a Laboratory Manual, 2nd Edn. Cold Spring Harbor Laboratory Press);

transcription labeling using RNA polymerase (Melton, D A, Krieg, P A, Rebagkiati, M R, Maniatis, T, Zinn, K, Green, M R. (1984) Nucleic Acid Res., 12, 7035-7056); and

non-isotopic labeling of DNA by incorporating modified nucleotides (Kricka, L J. (1992) Nonisotopic DNA Probing Techniques. Academic Press).

For testing for an allergic disease using hybridization techniques, for example, Northern hybridization, dot blot hybridization, or DNA microarray technique may be used. Furthermore, gene amplification techniques, such as RT-PCR method may be used. By using the PCR amplification monitoring method during the gene amplification step in RT-PCR, one can achieve more quantitative analysis for the gene expression of the present invention.

In the PCR gene amplification monitoring method, the detection target (DNA or reverse transcript of RNA) is hybridized to probes that are dual-labeled at both ends with different fluorescent dyes whose fluorescences cancel each other out. When the PCR proceeds and Taq polymerase degrades the probe with its 5′-3′ exonuclease activity, the two fluorescent dyes become distant from each other and the fluorescence becomes to be detected. The fluorescence is detected in real time. By simultaneously measuring a standard sample in which the copy number of the target is known, it is possible to determine the copy number of the target in the subject sample with the cycle number where PCR amplification is linear (Holland, P. M. et al., 1991, Proc. Natl. Acad. Sci. USA 88: 7276-7280; Livak, K. J. et al., 1995, PCR Methods and Applications 4(6): 357-362; Heid, C. A. et al., 1996, Genome Research 6: 986-994; Gibson, E. M. U. et al., 1996, Genome Research 6: 995-1001). For the PCR amplification monitoring method, for example, ABI PRISM7700 (PE Biosystems) may be used.

The method of testing for an allergic disease in the present invention can be also carried out by detecting a protein encoded by the indicator gene. Hereinafter, a protein encoded by the indicator gene is described as an indicator protein. For such test methods, for example, Western blotting method, immunoprecipitation method, and ELISA method may be employed using antibody that binds to the indicator protein.

Antibodies that bind to the indicator protein used in the detection may be produced by techniques known to those skilled in the art. Antibodies used in the present invention may be polyclonal or monoclonal antibodies (Milstein, C. et al., 1983, Nature 305 (5934): 537-40). For example, polyclonal antibody against an indicator protein may be produced by collecting the blood from mammals sensitized with the antigen, and separating the serum from this blood using known methods. As a polyclonal antibody, the serum containing polyclonal antibody as such may be used. As the occasion demands, a fraction containing polyclonal antibody can be further isolated from this serum. Also, monoclonal antibody may be obtained by isolating immune cells from mammals sensitized with the antigen, fusing these cells with myeloma cells, and such, cloning hybridomas thus obtained, and collecting the antibody as a monoclonal antibody from the culture of the hybridomas.

For detecting an indicator protein, these antibodies may be appropriately labeled. Alternatively, instead of labeling the antibody, a substance that specifically binds to the antibody, for example, protein A or protein G, may be labeled to arrange an indirect detection of indicator protein. More specifically, one example of an indirect detection method is ELISA.

Protein or its partial peptide used as an antigen may be obtained, for example, by inserting the gene or its portion into an expression vector, introducing it into an appropriate host cell to produce a transformant, culturing the transformant to express the recombinant protein, and purifying the expressed recombinant protein from the culture or the culture supernatant. Alternatively, amino acid sequences encoded by these genes, or oligopeptides comprising portions of the amino acid sequence encoded by the full-length cDNA are chemically synthesized to be used as the antigen.

Furthermore, in the present invention, a testing for an allergic disease can be performed using not only the expression level of an indicator gene but also the activity of an indicator protein in the biological sample as an index. Activity of an indicator protein means a biological activity intrinsic to each protein. The detection of activity of an indicator protein can be achieved by known method.

Carboxypeptidase M (Tan, F.; Chan, S. J.; Steiner, D. F.; Schilling, J. W.; Skidgel, R. A.; Molecular cloning and sequencing of the cDNA for human membrane-bound carboxypeptidase M: comparison with carboxypeptidases A, B, H, and N. J. Biol. Chem. 264: 13165-13170, 1989) has been cloned from a cDNA library of human placenta, has an open reading frame of 1317 bp, and encodes 439 amino acids. It is expressed in many tissues and cultured cells as a membrane bound carboxypeptidase. It has been reported that this is expressed during differentiation from a monocyte to a macrophage, but its relation to allergic diseases is not known. Carboxypeptidase M is a protease that selectively cleaves basic amino acids at the C-terminal end. Therefore, based on this enzyme reaction, activity of carboxypeptidase M can be measured biologically. Examples of basic amino acids are arginine and lysine.

Cathepsin C (Paris, A.; Strukelj, B.; Pungercar, J.; Renko, M.; Dolenc, I.; Turk, V.: Molecular cloning and sequence analysis of human preprocathepsin C. FEBS Lett. 369: 326-330, 1995) is one of the proteases found in animal cells, also called dipeptidylpeptidase I. Its representative substrate is glycyl-L-phenylalaninamide. Therefore, the biological activity of cathepsin C can be found out by measuring the digestion activity of this substrate compound. Besides, cathepsin C is known to catalyze hydrolysis of an amide bond near pH 5 and to catalyze transfer reaction near pH 7 in which an amino group of a different substrate molecule is an acceptor.

Since endothelin-A receptors (Maggi, M.; Barni, T.; Fantoni, G.; Mancina, R.; Pupilli, C.; Luconi, M.; Crescioli, C.; Serio, M.; Vannelli, G. B.; Expression and biological effects of endothelin-1 in human gonadotropin-releasing hormone-secreting neurons. J. Clin. Endocr. Metab. 85: 1658-1665, 2000) are receptor proteins, signal transduction into a cell due to binding of a ligand that can bind to these receptors can be referred to as the biological activity of each of these proteins. Signal transduction due to ligand binding can be detected by using as indicators, elevation of calcium concentration in a cell, change in cell morphology induced as a result of signal transduction, and such.

Osteoblast specific factor 2 (OSF2os; Horiuchi K, Amizuka N, Takeshita S, Takamatsu H, Katsuura M, Ozawa H, Toyama Y, Bonewald L F, Kudo A.; Identification and characterization of a novel protein, periostin, with restricted expression to periosteum and periodontal ligament and increased expression by transforming growth factor beta. J Bone Miner Res. 1999 July; 14(7):1239-49) is a 90 kDa protein. From differences in length at the C-terminal side due to alternative splicing, existence of 4 transcription products have been elucidated. It is now called periostin. It is expressed in the bones and some expression is found in the lungs. It is thought to be involved in cell adhesion during osteogenesis. It is highly homologous to betaig-h3, which is induced by TGFβ, and periostin itself is also induced by TGFβ in primary osteoblast cells. Its detection is possible using strengthened expression of bone specific genes as an indicator.

DD96 (Kocher O, Comella N, Tognazzi K, Brown L F.; Identification and partial characterization of PDZK1: a novel protein containing PDZ interaction domains. Lab Invest. 1998 January; 78(1):117-25) is a gene that is cloned from the kidney and encodes a 17 kD membrane bound protein. It is also called MAP17 and expression in cancer cells, cornified cells, and epithelial cells have been recognized, but its detailed function is not known.

CYP1B1 (Sutter, T. R.; Tang, Y. M.; Hayes, C. L.; Wo, Y. -Y. P.; Jabs, E. W.; Li, X.; Yin, H.; Cody, C. W.; Greenlee, W. F.: Complete cDNA sequence of a human dioxin-inducible mRNA identifies a new gene subfamily of cytochrome P450 that maps to chromosome 2. J. Biol. Chem. 269: 13092-13099, 1994) is one type of dioxin-inducible P450. P450 involved in metabolism of dioxins are involved in ring hydroxylation and cleavage, dehalogenation, glucuronidation, and such of the molecular structure of dioxin. Among them, CYP1B1 is a metabolic enzyme that metabolizes 2,3,7,8-TCDD. Examples of metabolites include a compound generated by Cl-elimination and hydroxylation of 2,3,7,8-TCDD. Therefore, CYP1B1 can be detected enzymatically using this enzyme activity as an indicator.

Normally, in the testing method of this invention, a biological sample from a subject is used as the sample. Respiratory tract epithelial cells and such may be used as the biological sample. Method to obtain respiratory tract epithelial cells is well known. That is, a sample can be obtained under a bronchoscope by physical detachment using forceps. The obtained sample is prepared by freezing, by formalin fixation, and by cultivation in a media. Furthermore, as the biological sample in the present invention, blood, sputum, secretion from nasal mucosa, bronchoalveolar lavage fluid, lung scrape, and such may be used. These biological samples are collected using known methods.

When the biological sample is cells of respiratory tract epithelial cells and such, samples for immunological measurements of the aforementioned proteins can be made by preparing a lysate. Otherwise, samples for measuring mRNA corresponding to the aforementioned genes can be made by extracting mRNA from this lysate. For extraction of lysate and mRNA of the biological sample, it is useful to utilize a commercially available kit. Otherwise, biological samples in the liquid form such as blood, nasal mucous secretion, and bronchoalveolar lavage fluid can be made into samples for measurement of proteins and genes by diluting with buffer and such, as necessary.

The measured value of expression level of indicator genes in respiratory tract epithelial cells can be corrected by known methods. As a result of correction, change in gene expression level in cells can be compared. Based on the measured value of expression level of genes that are expressed in the respiratory tract epithelial cells and do not show large fluctuations in their expression level regardless of the condition of the cell (housekeeping genes), correction of the measured value is performed by correcting the measured value of expression level of the genes that are to be used as indicators in this invention.

The indicator genes of this invention showed increase of expression level in a plurality of respiratory tract epithelial cell lines stimulated with IL-4 or IL-13. Therefore, testing for allergic diseases such as bronchial asthma can be performed using the expression level of indicator genes as indicators.

Test for an allergic disease in the present invention includes, for example, the tests as described below. Even a patient who, in spite of manifestation of bronchial asthma, can be hardly diagnosed with an allergic disease by conventional tests can be easily judged to be an allergic disease patient by carrying out the tests based on this invention. More specifically, the increase in the expression level of the indicator gene in a patient showing symptoms suspected of allergic disease indicates a high possibility that the symptoms are caused by an allergic disease. There are two types of bronchial asthma, one type being caused by an allergic reaction, and the other type not. Since treatments for two types are completely different, diagnosis as to which type causes the bronchial asthma is a very important step in the treatment. The test method of this invention can provide an extremely important information in identifying causes of bronchial asthma.

Otherwise, tests to judge whether the allergic symptom is improving become possible. The indicator genes of this invention showed increase in expression level in the respiratory tract epithelial cells stimulated with IL-4 or IL-13. In bronchial asthma, respiratory tract epithelial tissue is a tissue that shows remarkable lesion. Therefore, genes whose expression varies in respiratory tract epithelial cells simulated with IL-4 or IL-13, which are cytokines that strongly induce allergic reactions, are useful for judging therapeutic effects. More specifically, elevation of expression of indicator genes in patients diagnosed with an allergic disease indicates that there is a strong possibility that the allergic symptom is progressing.

Additionally, this invention relates to use of a transgenic non-human animal in which expression level of indicator genes in the respiratory tract epithelial cells is elevated, as an animal model for an allergic disease. Animal model for an allergic disease is useful for elucidating changes of bronchial asthma in vivo. Furthermore, the animal model for an allergic disease of this invention is useful for evaluating therapeutic agents for allergic bronchial asthma.

Because of this invention, it was elucidated that the expression levels of the aforementioned indicator genes in respiratory tract epithelial cells rise due to stimulation by IL-4 or IL-13. Therefore, animals in which expression levels of these genes or genes functionally equivalent to these genes are artificially enhanced in respiratory tract epithelial cells can be used as animal models for allergic diseases. Elevation of expression level in respiratory tract epithelial cells includes elevation of expression level of target genes in the entire respiratory tract tissue. That is, elevation of expression level of the aforementioned genes includes elevation of the expression level of the genes in the entire respiratory tract tissue or in the entire body, in addition to the elevation in the respiratory tract epithelia alone.

The functionally equivalent genes used in this invention are genes that encode proteins having activity similar to the known activity of proteins encoded by each of the indicator genes. A representative example of functionally equivalent genes includes a counterpart of indicator genes of a transgenic animal that intrinsically has the counterpart.

Otherwise, genes that encode proteins having for example, sequence homology of 90% or more, preferably 95% or more, and more preferably 99% or more, towards amino acid sequence of the aforementioned indicator proteins can be shown to be genes that are functionally equivalent to the aforementioned indicator genes. Additionally, genes (1) that can be amplified using, as primers, oligonucleotides comprising the nucleotide sequence of carboxypeptidase M, cathepsin C, endothelin-A receptor, osteoblast specific factor 2, DD96(MAP17), and CYP1B1, which were used in the Examples, and (2) that encode proteins whose expression level significantly increases in respiratory tract epithelial cells stimulated with IL-4 and IL-13 are functionally equivalent genes.

Genes whose expression increase due to IL-4 or IL-13 stimulation can be said to be genes included in the signal transduction pathway of these cytokines. In other words, IL-4 or IL-13 stimulation can be considered to be expressed as allergic symptoms through enhancement of expression of these genes. That is, genes that increase their expression due to IL-4 or IL-13 stimulation can be said to be genes that accomplish important roles in pathological formation of allergies in the respiratory tract epithelium. Therefore, in treatment of allergies, drugs that suppress the expression of these genes or inhibit their activities are expected to have the effect of not only simply improving allergic symptoms but also removing the fundamental cause of pathological formation of allergies.

As described above, a gene the expression level of which in respiratory tract epithelial cells is increased upon stimulation thereof with IL-4 or IL-13 is very important. Therefore, it is highly significant to assess the role of the gene and effects of drugs targeting this gene using transgenic animals, which can be obtained by elevating the expression level of this gene in vivo, as the allergic disease model animal.

Allergic disease model animals according to the present invention are useful in not only screening for drugs for treating or preventing allergic diseases as described below but also elucidating mechanisms of allergic diseases, furthermore, testing the safety of compounds screened.

For example, if allergic disease model animals according to the present invention either develop clinical manifestations of bronchial asthma or show changes in measured values related to any allergic diseases, it is possible to construct a screening system for searching for a compound having activity to recover normal conditions.

In the present invention, increase in the expression level means the state wherein a target gene is transduced as a foreign gene and forcibly expressed; the state wherein transcription of a gene inherent in the host and translation thereof into protein are increased; or the state wherein decomposition of the translation product, protein, is suppressed. Gene expression level can be confirmed by, for example, the quantitative PCR as described in Examples. Furthermore, activity of translation product, protein, can be confirmed by comparing to that in the normal state.

A typical transgenic animal is the one to which a gene of interest is transduced to be forcibly expressed. Examples of another type of transgenic animals are those in which a mutation is introduced into the coding region of the gene to increase its activity or to modify the amino acid sequence of the gene product protein so as to be hardly decomposed. Examples of mutation in the amino acid sequence are the substitution, deletion, insertion, or addition of amino acid(s). In addition, by mutagenizing the transcriptional regulatory region of the gene, the expression itself of the gene of this invention can be controlled.

Methods for obtaining transgenic animals with a particular gene as a target are known. That is, a transgenic animal can be obtained by a method wherein the gene and ovum are mixed and treated with calcium phosphate; a method wherein the gene is introduced directly into the nucleus of oocyte in pronuclei with a micropipette under a phase contrast microscope (microinjection method, U.S. Pat. No. 4,873,191); or a method wherein embryonic stem cells (ES cells) are used. Furthermore, there have been developed a method for infecting ovum with a gene-inserted retrovirus vector, a sperm vector method for transducing a gene into ovum via sperm, or such. Sperm vector method is a gene recombination technique for introducing a foreign gene by fertilizing ovum with sperm after a foreign gene has been incorporated into sperm by the adhesion or electroporation method, etc. (M. Lavitranoet, et al. Cell, 57, 717, 1989).

Transgenic animals used as the allergic disease model animal of the present invention can be produced using all the vertebrates except for humans. More specifically, transgenic animals having various transgene and being modified gene expression levels thereof are produced using vertebrates such as mice, rats, rabbits, miniature pigs, goats, sheep, or cattle.

Furthermore, the present invention relates to a method of screening for a candidate compound for a therapeutic agent for an allergic disease. In this invention, the indicator gene shows a significant increase in its expression level in respiratory tract epithelial cells stimulated with IL-4 or IL-13. Therefore, it is possible to obtain a therapeutic agent for an allergic disease by selecting a compound capable of reducing the expression level of such a gene. Compounds that reduce the expression level of a gene are those having inhibitory effects on any steps of the transcription or translation of a gene, or the activity expression of a protein.

A method of screening for a therapeutic agent for an allergic disease of this invention can be carried out either in vivo or in vitro. This screening method can be carried out, for example, according to the steps as described below. The indicator gene in the screening method of this invention includes, in addition to the genes described as indicator genes above, any genes functionally equivalent thereto. The steps of the screening method are:

(1) administering a candidate compound to a test animal;

(2) measuring the expression level of the indicator gene in a biological sample from the test animal; and

(3) selecting a compound that reduces the expression level of the indicator gene, compared to a control.

As a test animal in the screening method of the present invention, for example, an allergic disease model animal, a transgenic animal in which a human indicator gene is forcibly expressed, may be used. When a promoter whose transcription activity is controlled by a substance such as an appropriate drug is used in the expression vector, the expression level of the foreign indicator gene can be adjusted by administering said substance to the transgenic animal.

Thus, by administering a drug candidate compound to a model animal in which an indicator gene is forcibly expressed, and monitoring the action of the compound toward the expression of the indicator gene in the biological sample from the model animal, effects of the drug candidate compound on the expression level of the indicator gene can be detected. Changes in the expression levels of the indicator gene in biological samples from test animals can be monitored by a method similar to above-described testing method of this invention. Furthermore, based on the detection results, by selecting compounds that reduce the expression level of the indicator gene, drug candidate compounds can be screened.

More specifically, the screening according to the present invention can be carried out by comparing the expression level of the indicator gene in the biological sample collected from a test animal to that in a control. As a biological sample, smooth muscle cells, cornified cells, nasal mucosa epithelial cells, intestinal epithelial cells, lymphocytes, mast cells, eosinophils, basophils, neutrophils, and such can be used. Methods for collecting and preparing these biological samples are known.

These screening methods enable the selection of drugs involved in the expression of indicator genes in various ways. More specifically, for example, drug candidate compounds having the following action points can be found:

activation of signal transduction pathway to induce the expression of an indicator gene;

elevation of the transcription activity of the indicator gene; and

stabilization of the transcription product of the indicator gene or inhibition of the decomposition thereof, etc.

Examples of in vitro screening include a method in which cells expressing an indicator gene are contacted with a candidate compound to select a compound that reduces the expression level of the indicator gene. This screening may be carried out, for example, according to the steps of:

(1) contacting a candidate compound with cells expressing an indicator gene;

(2) measuring the expression level of the indicator gene; and

(3) selecting a compound that reduces the expression level of the candidate gene, compared to a control.

In the present invention, cells expressing an indicator gene can be obtained by inserting the indicator gene to an appropriate expression vector, and introducing said vector into a suitable host cell. Any vectors and host cells may be used as long as they are able to express the gene of this invention. Examples of host cells in the host-vector system are Escherichia coli, yeast, insect cells, animal cells, and such, and vectors usable for respective host cells can be appropriately selected.

Vectors may be introduced into the host by the biological method, physical method, chemical method, etc. Examples of the biological method are a method using virus vectors, a method using a specific receptor, cell-fusion method (HVJ (Sendai virus) method, polyethylene glycol (PEG) method, electric cell fusion method, microcell-mediated chromosome transfer. Examples of the physical method are a microinjection method, electroporation method, and a method using the gene particle gun (gene gun). Examples of the chemical method are a calcium phosphate precipitation method, liposome method, DEAE-dextran method, protoplast method, erythrocyte ghost method, erythrocyte membrane ghost method, and microcapsule method.

As cells in which an indicator gene expresses, the human lung cancer cell A549 and human bronchial epithelial cell BEAS-2B are preferable for the screening method of the present invention. These cells are commercially available from ATCC.

In the screening method of this invention, first a candidate compound is added to the cell strain. Then, the expression level of an indicator gene in the cell strain is measured to select a compound that reduces the expression level of the gene.

In the screening method of this invention, expression levels of indicator genes can be compared not only based on the expression levels of proteins encoded by these genes but also based on the corresponding mRNAs detected. For performing the comparison of expression levels using mRNA, the process for preparing mRNA sample as described above is carried out in place of the process for preparing protein samples. Detection of mRNA and protein can be performed by known methods as described above.

Furthermore, based on the disclosure of this invention, it is possible to obtain the transcriptional regulatory region for the indicator gene of this invention and construct a reporter assay system. Reporter assay system means a system for screening for a transcriptional regulatory factor that acts on the transcriptional regulatory region using the expression level of a reporter gene localized downstream of the transcriptional regulatory region as an index.

That is, this invention relates to a method of screening for candidate compounds for a therapeutic agent for an allergic disease, in which the indicator genes are one or more genes selected from the group consisting of carboxypeptidase M, cathepsin C, endothelin-A receptor, osteoblast specific factor 2, DD96(MAP17), CYP1B1, and genes that are functionally equivalent to these genes, the method comprising the following steps:

(1) contacting a candidate substance with cells transfected with a vector containing a transcription regulatory region of one or more indicator genes and a reporter gene that is expressed under the control of the transcription regulatory region,

(2) measuring the activity of the aforementioned reporter gene, and

(3) selecting a compound that lowers the reporter gene expression level, compared to a control.

Examples of transcription regulatory regions are promoters, enhancers, and furthermore, CAAT box and TATA box, which are normally seen in the promoter region. Also, as reporter genes, CAT (chloramphenicol acetyltransferase) gene, luciferase gene, growth hormone genes, and such may be used. In not a few number of the indicator genes of this invention, a transcription regulatory region has already been elucidated.

Otherwise, the transcription regulatory region of the indicator genes of this invention can be obtained as follows. That is, first, screening is performed by a method that uses PCR or hybridization based on the nucleotide sequences of the indicator genes disclosed in this invention, and a genomic DNA clone containing the cDNA sequence is obtained from a human genome DNA library such as BAC library and YAC library. Based on the obtained genomic DNA sequence, the transcription regulatory region of cDNA disclosed in this invention is estimated, and the transcription regulatory region is obtained. A reporter construct is constructed by cloning the obtained transcription regulatory region so that it is positioned upstream of the reporter gene. The obtained reporter construct is transfected into a cultured cell strain and is made into a transformant for screening. By contacting the candidate compounds with this transformant, screening of compounds that regulate the expression of reporter genes can be performed.

As the screening method of this invention in vitro, a screening method based on activities of indicator proteins can be used. That is, this invention relates to a method of screening for a therapeutic agent for an allergic disease, in which the indicator genes are one or more genes selected from the group consisting of carboxypeptidase M, cathepsin C, endothelin-A receptor, osteoblast specific factor 2, DD96(MAP17), CYP1B1, and genes that are functionally equivalent to these genes, the method comprising the following steps:

(1) contacting the candidate substance with one or more proteins encoded by the indicator genes,

(2) measuring the activity of the aforementioned proteins,

(3) selecting a compound that lowers the activity of the aforementioned proteins, compared to a control.

Each of the activities possessed by the indicator proteins of this invention, which are carboxypeptidase M, cathepsin C, endothelin-A receptor, osteoblast specific factor 2, DD96(MAP17), and CYP1B1, has been already mentioned. Using these activities as indicators, compounds having activity to inhibit these activities can be screened. Compounds that can be obtained in this manner suppress the function of carboxypeptidase M, cathepsin C, endothelin-A receptor, osteoblast specific factor 2, DD96(MAP17), and CYP1B1. As a result, bronchial asthmatic attack can be regulated through inhibition of indicator proteins whose expression is induced in the respiratory tract epithelial cells.

Candidate test compounds used in such screening include, in addition to compound preparations synthesized by existing chemical methods such as steroid derivatives and compound preparations synthesized by combinatorial chemistry, mixtures of multiple compounds such as extracts from animal or plant tissues, or microbial cultures, and their purified preparations, etc.

Polynucleotide, antibody, cell strain, or model animal necessary for various screening methods according to this invention can be previously combined into a kit. More specifically, for example, a kit may be composed of a cell expressing an indicator gene and a reagent to measure the expression level of the indicator gene. As a reagent for measuring the expression level of an indicator gene, for example, a polynucleotide containing the nucleotide sequence of at least one indicator gene, or an at least 15-nucleotide-long oligonucleotides containing a nucleotide sequence complementary to the complementary strand thereof can be used. Alternatively, antibody that recognizes a peptide containing the amino acid sequence of at least one indicator protein may be used as a reagent. In these kits may be packaged a substrate compound used for the detection of the indicator, medium and vessel for cell culturing, positive and negative standard samples, and furthermore, a manual describing how to use the kit.

The compounds selected by the screening method of this invention are useful as a therapeutic agent for an allergic disease. Also, the antisense DNA that can suppress the expression of an indicator gene, and, furthermore, antibody recognizing a protein encoded by an indicator gene are also useful as the therapeutic agent for an allergic disease. The therapeutic agent for an allergic disease according to this invention can be formulated by including the compound selected by the screening method as the effective ingredient, and mixing with a physiologically acceptable carrier, excipient, diluent, or the like. Aiming at the amelioration of allergic symptoms, the therapeutic agent for an allergic disease of this invention can be administered orally or parenterally.

Oral drugs can take any dosage forms selected from a group of granule, powder, tablet, capsule, solution, emulsion, suspension, etc. Injections can include the subcutaneous injection, intramuscular injection, intraperitoneal injection, etc.

Furthermore, for administering the compound that is composed of protein, the therapeutic effect can be achieved by introducing a gene encoding the protein into the living body using gene therapeutic techniques. The techniques for treating disease by introducing a gene encoding a therapeutically effective protein into the living body and expressing it therein are known.

Alternatively, the antisense DNA can be incorporated downstream of an appropriate promoter sequence to be administered as an antisense RNA expression vector. When this expression vector is introduced into T cells of an allergic disease patient, the therapeutic effect on allergic disease can be achieved by reducing the expression level of the gene through the expression of corresponding antisense gene. For introducing the expression vector into T cells, methods performed either in vivo or ex vivo are known.

Although the dosage may vary depending on the age, sex, body weight, and symptoms of a patient, treatment effects, method for administration, treatment duration, type of active ingredient contained in the drug composition, or such, it can be usually administered in the range of 0.1 mg˜500 mg, preferably 0.5 mg˜20 mg per dose for an adult. However, since the dosage varies according to various conditions, amount less than the above-described dosage may be sufficient in some cases, and dosage exceeding the above-described range may be required in other cases.

Any prior art literatures cited herein are incorporated by reference.

### BRIEF DESCRIPTION OF THE DRAWINGS

FIG. 1 is a set of graphs showing the results of measuring the expression levels of the carboxypeptidase M gene in cultured bronchial epithelial cells stimulated with IL-4 and IL-13, or with other cytokines. Those on the left are results corrected with β-actin gene, and those on the right are results corrected with GAPDH gene. Graphs on the top row indicate the relative expression level (relative ratio of control) in each lot of cells when stimulated with IL-4 (center) and IL-13 (right) to the control (left). Graphs in the middle row show change in expression level with time, 0, 6, 12, 24, and 48 hours after treatment. The numbers on the abscissa indicate cultivation time. Graphs in the bottom row show the change in expression level 24 hours after treatment with other cytokines.

FIG. 2 is a set of graphs showing the results of measuring the expression levels of the cathepsin C gene in cultured bronchial epithelial cells stimulated with IL-4 and IL-13, or with other cytokines. Each graph shows analogous content to that of each graph in FIG. 1.

FIG. 3 is a set of graphs showing the results of measuring the expression levels of the endothelin-A receptor gene in cultured bronchial epithelial cells stimulated with IL-4 and IL-13, or with other cytokines. Each graph shows analogous content to that of each graph in FIG. 1.

FIG. 4 is a set of graphs showing the results of measuring the expression levels of the osteoblast specific factor gene in cultured bronchial epithelial cells stimulated with IL-4 and IL-13, or with other cytokines. Each graph shows analogous content to that of each graph in FIG. 1.

FIG. 5 is a set of graphs showing the results of measuring the expression levels of the DD96(MAP17) gene in cultured bronchial epithelial cells stimulated with IL-4 and IL-13, or with other cytokines. Each graph shows analogous content to that of each graph in FIG. 1.

FIG. 6 is a set of graphs showing the results of measuring the expression levels of the CYP1B1 gene in cultured bronchial epithelial cells stimulated with IL-4 and IL-13, or with other cytokines. Each graph shows analogous content to that of each graph in FIG. 1.

### BEST MODE FOR CARRYING OUT THE INVENTION

The present invention will be explained in detail below with reference to examples, but it is not to be construed as being limited thereto.

### EXAMPLE 1

Selection of Candidate Genes Using DNA Microarray

1. Cultivation of Normal Human Bronchial Epithelial Cells, and IL-4 or IL-13 Stimulation

Three lots of normal human bronchial epithelial cells available from Clonetics were purchased (8F1756, 8F1548, 8F1805). Cells (5×10⁵) contained in one vial were divided into three equal parts (1.67×10⁵/75 cm² flask) for no stimulation, IL-4 stimulation, and IL-13 stimulation, and these were cultivated for approximately 8 to 10 days in SABM media (Clonetics) with medium exchange. During this procedure, BPE (bovine pituitary extract), hydrocortisone, hEGF, epinephrine, transferrin, insulin, retinoic acid, BSA-FAF, triiodothyronine, GA-1000 (gentamicin/amphotericin-B) were added to the media according to the attached protocol.

Before cytokine stimulation, the cells were washed with PBS, and then placed into SABM without added factors. IL-4 (10 ng/mL) and IL-13 (50 ng/mL) (both from Peprotech) were added thereto, and this was cultivated for 24 hours. Observation of changes with passage of time (0, 6, 12, 24, and 48 hours) was carried out in a similar manner.

2. Other Cytokine Stimulation of Normal Human Bronchial Epithelial Cells

Using cells from lot 8F1548, cultivation was performed similarly to that of 1. In place of IL-4 and IL-13, 50 ng/mL of TNFα, IL-1β, IL-5, IL-6, and IL-9 (all from Peprotech) were added and cultivated for 24 hours.

3. Preparation of RNA for GeneChip

Respiratory tract epithelial cells treated as mentioned above were dissolved in Isogen (Nippon Gene; Wako Pure Chemicals), and from this solution, RNA was separated according to a protocol attached to Isogen. After addition of chloroform, this was agitated, then centrifuged, and its aqueous layer was collected. Next, isopropanol was added, this was agitated, and then centrifuged to collect the precipitated total RNA.

4. cDNA Synthesis for GeneChip

Single stranded cDNA was prepared from 5 μg of total RNA, which was prepared from cells of lot 8F1756, by reverse transcription using Superscript II Reverse Transcriptase (Life Technologies) following the method of Expression Analysis Technical Manual by Affymetrix, and by using T7-(dT)₂₄ (Amersham Pharmacia) as a primer. The T7-(dT)₂₄ primer comprises a nucleotide sequence in which d(T)₂₄ is added to a T7 promoter nucleotide sequence, as shown below.

T7-(dT)₂₄ primer (SEQ ID NO: 1) 5′-GGCCAGTGAATTGTAATACGACTCACTATAGGGAGGCGG-(dT)₂₄-3′

Next, according to Expression Analysis Technical Manual, DNA ligase, DNA polymerase I, and RNase H were added to synthesize double stranded cDNA. After phenol-chloroform extraction of cDNA, this was passed through Phase Lock Gels, and was purified by ethanol precipitation.

Furthermore, using BioArray High Yield RNA Transcription Labeling Kit, biotin-labeled cRNA was synthesized. Using RNeasy Spin column (QIAGEN), cRNA was purified and then fragmented by treatment with heat.

To a hybridization cocktail, 12.5 μg of this cRNA was added according to Expression Analysis Technical Manual. This was placed into an array and was hybridized for 16 hours at 45° C.

After the array was washed, streptavidin phycoerythrin was added for staining. After washing, a mixed antibody solution of normal goat IgG and biotinylated goat IgG was added to the array. Furthermore, in order to enhance fluorescence intensity, streptavidin phycoerythrin was added again for staining. After washing, this was set into a scanner and was analyzed by a GeneChip software.

5. GeneChip Analysis

Data analysis was performed using Suite, which is a GeneChip analysis software. Average Intensity (1) and Background Average (2) were investigated by Absolute Analysis, and 3 average values obtained for no stimulation, IL-4 stimulation, and IL-13 stimulation by subtracting (2) from (1) were used as scale factors for comparison analysis.

First, absolute analysis was performed to analyze one chip data. Positives and negatives were determined by comparing the fluorescence intensity of perfect match and mismatch of a probe set. Judgment into three categories of Absolute Calls, which are P (present), A (absent), and M (marginal), were made by values of Pos Fraction, Log Avg, and Pos/Neg.

Pos Fraction; ratio of positive pairs.

Log Avg; average of the log of fluorescence intensity ratio between probe cells of perfect match and mismatch.

Pos/Neg; ratio of the number of positive pairs and negative pairs.

Additionally, Average Difference (Avg Diff), which is the average value of the difference in fluorescence intensities between probe cells of perfect match and mismatch, was calculated for each gene.

Next, Comparison Analysis was performed on two sets of data. Comparisons were made between no stimulation and IL-4 stimulation, or between no stimulation and IL-13 stimulation, and differences in expression levels were ranked as follows. Judgment into 5 categories of difference calls, which are I, D, MI, MD, and NC, were made from values of Inc/Dec, Inc Ratio, Dpos-Dneg Ratio, and Log Avg Ratio Change.

Inc: Number of probe pairs that corresponded to IL-4 stimulation or IL-13 stimulation and no stimulation and that were judged to show increased expression levels for IL-4 stimulation or IL-13 stimulation.

Dec: Number of pairs judged to show decreased expression levels for IL-4 stimulation or IL-13 stimulation.

Inc/Dec: Ratio of the number of pairs judged to be Inc and number of pairs judged to be Dec.

Inc Ratio: Number of pairs judged to be Inc/number of pairs actually used.

Dpos/Dneg Ratio: Ratio between the number of Neg Change subtracted from that of Pos Change, and the number of pairs actually used.

Pos Change: Difference between the number of positive pairs in Absolute Analysis of IL-4 simulation or IL-13 stimulation, and the number of positive pairs in Absolute Analysis of no stimulation.

Neg Change: Difference between the number of negative pairs in Absolute Analysis of IL-4 simulation or IL-13 stimulation, and the number of negative pairs in Absolute Analysis of no stimulation.

Log Avg Ratio Change: Difference between Log Avg in Absolute Analysis of IL-4 stimulation or IL-13 stimulation and no stimulation.

Increased: I,

Decreased: D,

Marginally Increased: MI,

Marginally Decreased: MD, and

No Change: NC

Additionally, genes whose expression was altered (enhanced or reduced) due to IL-4 simulation or IL-13 stimulation by values of Fold Change, which is a ratio of Avg Diff in Absolute Analysis of no stimulation with IL-4 simulation or no stimulation with IL-13 stimulation, were selected. Table 1 shows the results of narrowing down expressed genes in respiratory tract epithelial cells by lot number.

TABLE 1

| | 8F1548 | | 8F1805 | | 8F1756 | |
|---|---|---|---|---|---|---|
| | 24.BEC IL-4 | 25.BEC IL-13 | 30.BEC IL-4 | 31.BEC IL-13 | 4.BEC-IL4 | 5.BEC-IL13 |
| Data 1 | | | | | | |
| Increase or decrease in [Diff Call] | 188 | 227 | 250 | 159 | 189 | 164 |
| Commonly Varying Genes | 89 | | 73 | | 89 | |
| Data 2 | | | | | | |
| >2 or <−2 in [Fold Change] | 108 | 108 | 124 | 114 | 106 | 101 |
| Commonly Varying Genes | 46 | | 46 | | 53 | |
| Data 3 | | | | | | |
| >3 or <−3 in [Fold Change] | 46 | 48 | 42 | 42 | 49 | 44 |

| | | | |
|---|---|---|---|
| Commonly Varying Genes | 25 | 20 | 25 |

In the table, “increase or decrease in [Diff Call]” shows the number of genes whose expression levels showed differences due to IL-4 stimulation or IL-13 stimulation. The genes whose expression levels showed differences include genes that were judged as marginally increased (or marginally decreased) in the aforementioned ranking. The number of genes in which differences were commonly seen with both IL-4 and IL-13 was indicated for each lot as “Commonly Varying Genes”. Furthermore, the number of genes in which increase or decrease of twofold or more (>2 or <−2), and increase or decrease of threefold or more (>3 or <−3) were observed was indicated as “[Fold Change]”.

As a result, the following 6 types of genes were selected as genes whose expression levels showed twofold or greater increase for both IL-4 stimulation and IL-13 stimulation and whose expression levels commonly showed increase for two or more lots. These genes are closely related to allergies, and their expression levels increase by twofold or more due to stimulation by both IL-4 and IL-13, which are allergy related cytokines.

carboxypeptidase M

cathepsin C

endothelin-A receptor

osteoblast specific factor 2 (OSF2os)

DD96(MAP17)

and dioxin-inducible cytochrome P450 (CYP1B1 )

### EXAMPLE 2

Confirmation of Expression Level of Candidate Genes

To quantitatively confirm the expression level of 6 genes selected in Example 1, cultivated respiratory tract epithelial cells (Clonetics) were used to further perform quantitative PCR by ABI 7700. Three lots, 8F1756, 8F1548, and 8F1805, were used for the cultivated cells. Primer and TaqMan probe used for measurements by ABI 7700 were designed by Primer Express (PE Biosystems) based on sequence information of each gene. The 5′-end of TaqMan probe is labeled with FAM (6-carboxy-fluorescein) and the 3′-end is labeled with TAMRA (6-carboxy-N,N,N′,N′-tetramethylrhodamine). Nucleotide sequences of oligonucleotides used for forward primer (F), reverse primer (R), and TaqMan probe (TP) of each gene are as shown below. Genbank Accession No. corresponding to the nucleotide sequence of each indicator gene is shown in parenthesis following the name.

Carboxypeptidase M 004970)

F: acagagacgtttgtcctctctgc (SEQ ID NO: 2)

R: atgccccagttgcttgaacac (SEQ ID NO: 3)

TP: ccctcgtggccagttacccatttgata (SEQ ID NO: 4)

Cathepsin C (x87212)

F: tctcagaccccaatcctaagcc (SEQ ID NO: 5)

R: ctgcaataaggtatgggaagcc (SEQ ID NO: 6)

TP: tcttgtagccagtatgctcaaggctgtgaa (SEQ ID NO: 7)

Endothelin-A receptor (d11151)

F: acctctgcgctcttagtgttgac (SEQ ID NO: 8)

R: gcagttaccaaaggaatcccaa (SEQ ID NO: 9)

TP: tacagagcagttgcctcctggagtcgtgtt (SEQ ID NO: 10)

Osteoblast specific factor 2 (d13666)

F: agcaaaccaccttcacggatc (SEQ ID NO: 11)

R: ggtgccagcaaagtgtattctcc (SEQ ID NO: 12)

TP: aattaggcttggcatctgctctgaggcc (SEQ ID NO: 13)

DD96(MAP17)(u21049)

F: gcctttgcagtcaaccacttctg (SEQ ID NO: 14)

R: tctgttcccaccaggactccat (SEQ ID NO: 15)

TP: atgatcctgaccgtcggaaacaaggc (SEQ ID NO: 16)

CYP1B1(u03688)

F: ttatgaagccatgcgcttct (SEQ ID NO: 17)

R: aagacagaggtgttggcagtg (SEQ ID NO: 18)

TP: cagctttgtgcctgtcactattcctcatg (SEQ ID NO: 19)

Total RNA extracted by the aforementioned method was treated with DNase (Nippon Gene). Then, cDNA, which was reverse transcribed using random hexamer (GIBCO BRL) as primer, was used as a template. For a standard curve to calculate the number of copies, a plasmid clone containing a nucleotide sequence region that is amplified by both primers was prepared for each of the genes, and this was diluted stepwise to be used as template for carrying out the reaction. The composition of reaction solution for monitoring PCR amplification is shown in Table 2.

TABLE 2
Composition of reaction in ABI-PRISM 7700 (Amount per well)

| Sterilized distilled water | 23.75 (μL) |
|---|---|
| 10x TaqMan buffer A | 5 |
| 25 mM MgCl₂ | 7 |
| dATP (10 mM) | 1.0 |
| dCTP (10 mM) | 1.0 |
| dGTP (10 mM) | 1.0 |
| dUTP (20 mM) | 1.0 |
| Forward Primer (10 μM) | 1.0 |
| Reverse Primer (10 μM) | 1.0 |
| TaqMan probe (2.0 μM) | 2.5 |
| AmpliTaq Gold (5 U/μL) | 0.25 |
| AmpErase UNG (1 U/μL) | 0.5 |
| Template solution | 5 |
| Total | 50 |

Additionally, to correct the differences of cDNA concentration in the sample, similar quantitative analysis was performed for β-actin gene and glyceraldehyde-3-phosphate dehydrogenase (GAPDH) gene as internal standards for correction. By correcting based on the number of copies of these genes, the number of copies of the genes of interest was calculated.

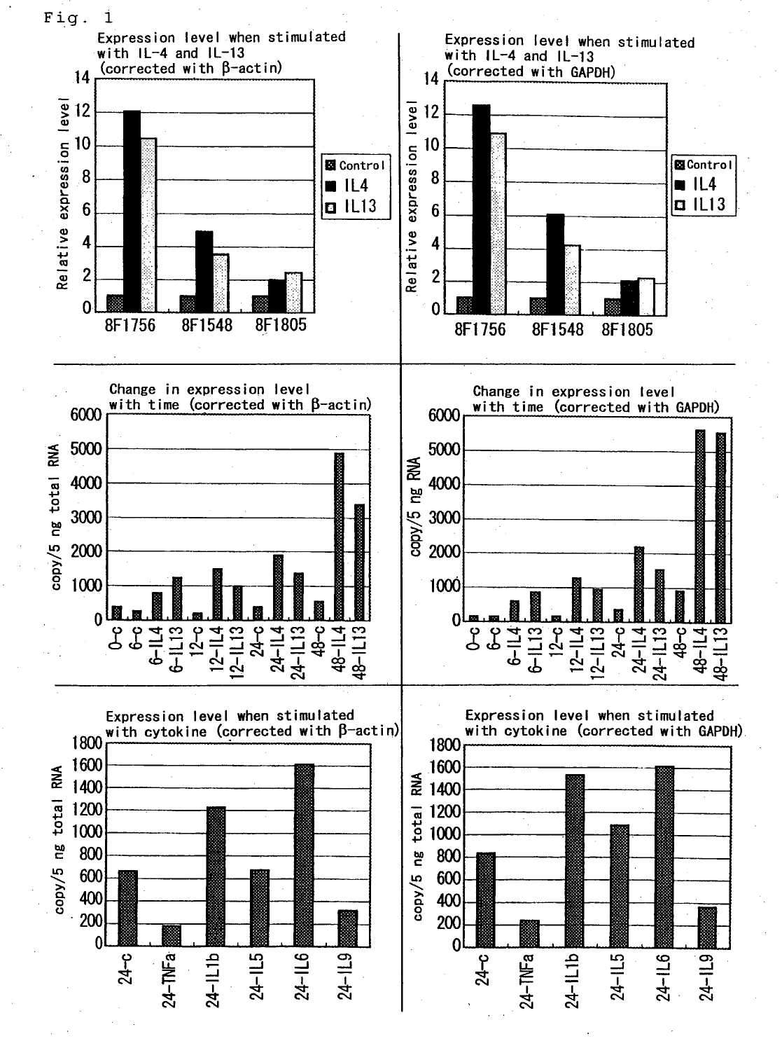

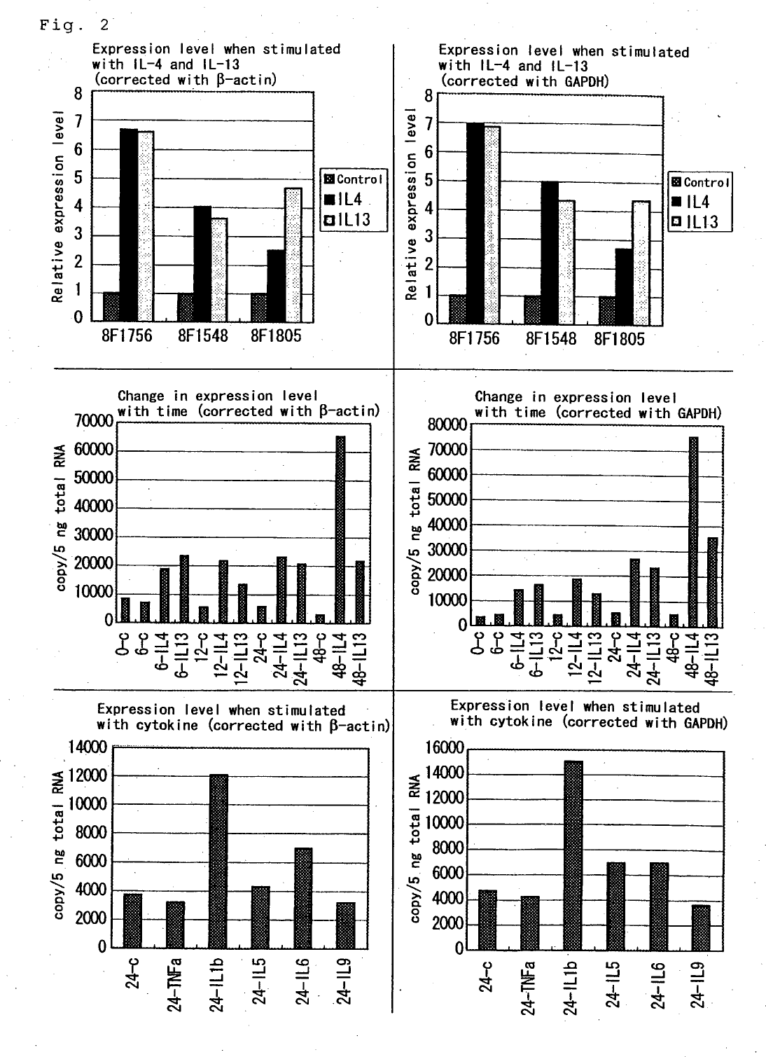

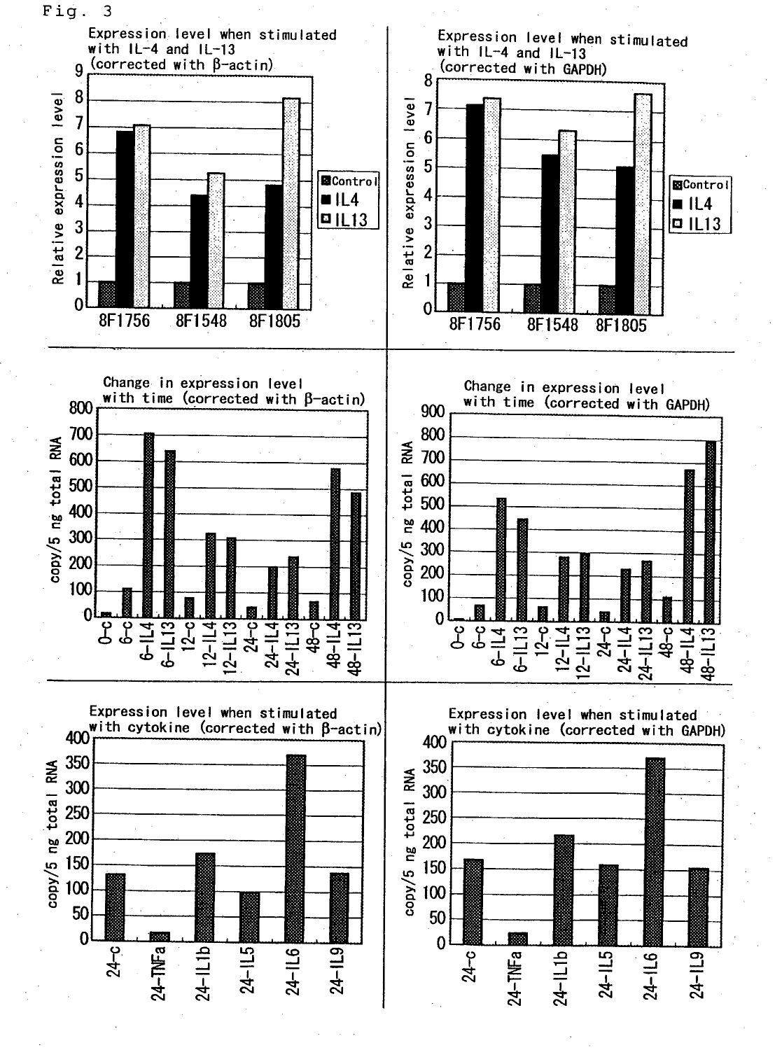

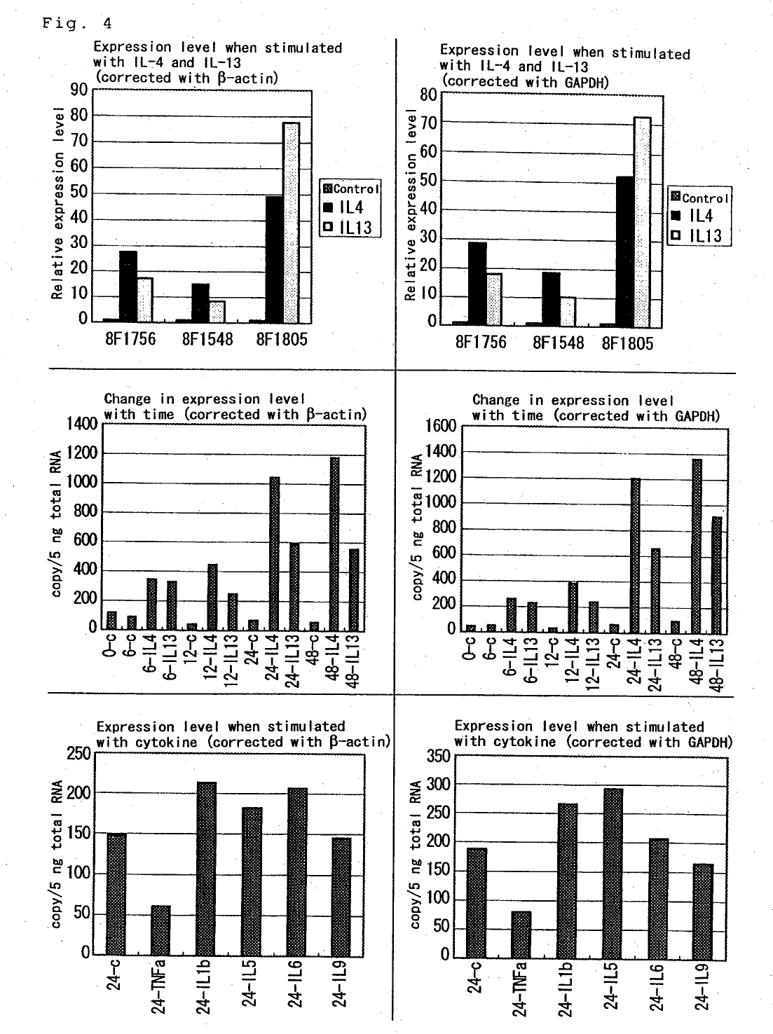

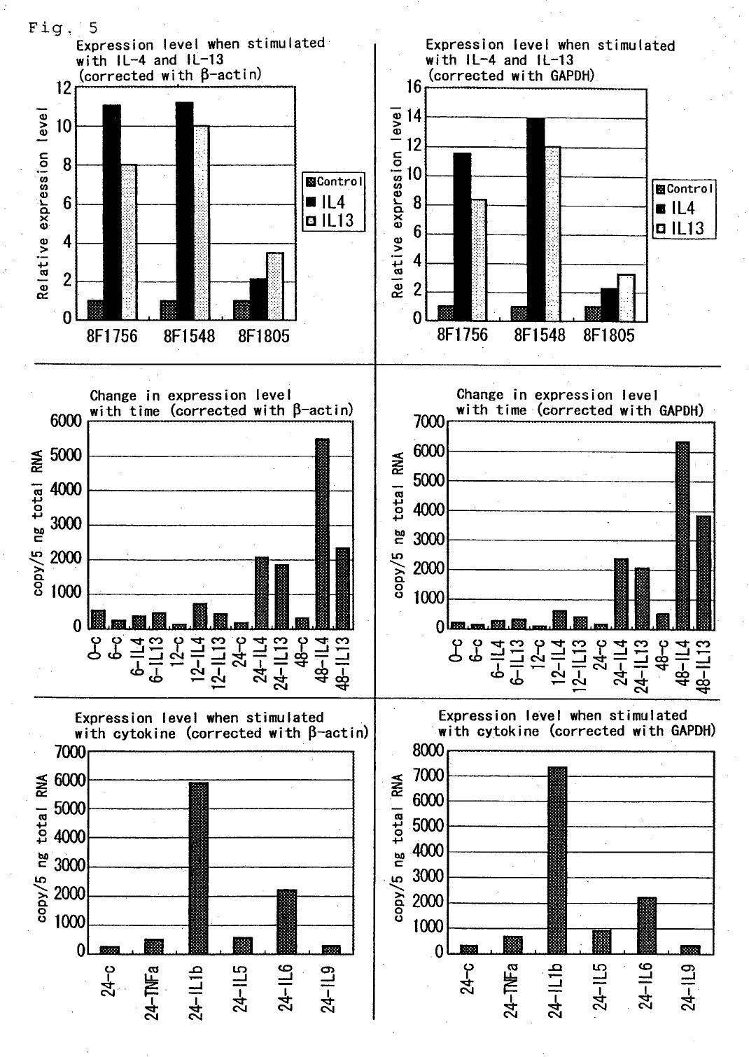

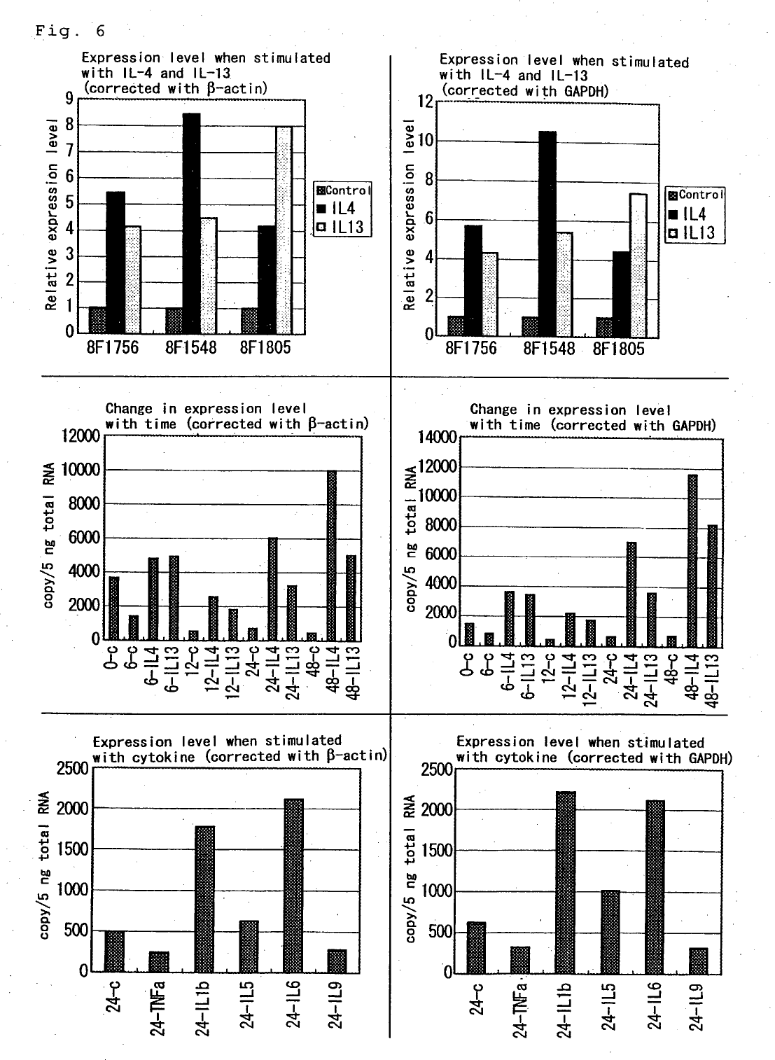

Primers and probes for measuring β-actin or GAPDH were those packaged with TaqMan β-actin Control Reagents (PE Biosystems). The nucleotide sequences are as shown below. The β-actin-corrected expression levels (copy/5 ng RNA) for each of the genes are shown in FIGS. 1 to 6.

β-actin forward primer (SEQ ID NO: 20)

TCA CCC ACA CTG TGC CCA TCT ACG A

β-actin reverse primer (SEQ ID NO: 21)

CAG CGG AAC CGC TCA TTG CCA ATG G

β-actin TaqMan probe (SEQ ID NO: 22)

(FAM)ATGCCC-T(TAMRA)-CCCCCATGCCATCCTGCGTp-3′

GAPDH forward primer (SEQ ID NO: 23)

GAAGGTGAAGGTCGGAGT

GAPDH reverse primer (SEQ ID NO: 24)

GAAGATGGTGATGGGATTTC

GAPDH TaqMan probe (SEQ ID NO: 25)

(FAM)CAAGCTTCCCGTTCTCAGCC(TAMRA)-3′

FAM: 6-carboxy-fluorescein

TAMRA: 6-carboxy-N,N,N′,N′-tetramethylrhodamine

As a result of quantitative PCR, the expression levels of 6 genes in the respiratory tract epithelial cells, where the genes were selected in Example 1, were elevated by twofold or more in three different respiratory tract epithelial cells due to IL-4 or IL-13 stimulation. Based on these results, elevation of expression levels of these indicator genes in response to IL-4 and IL-13 could be predicted in respiratory tract epithelial cells.

The indicator genes of this invention show common behavior among different lots of bronchial epithelial cells by IL-4 and IL-13 stimulation known to have close relationship to allergic reactions. Therefore, the indicator genes of this invention can be thought to be important genes regulating the progress of allergic reactions.

Industrial Applicability

By this invention, genes that increase their expression in respiratory tract epithelial cells stimulated with IL-4 or IL-13 were found. It is highly probable that genes whose expression elevates in respiratory tract epithelial cells stimulated with IL-4 or IL-13 are the fundamental causes of allergic symptoms in bronchial asthma. Therefore, the indicator genes provided by this invention become useful indicators for reliably knowing whether the bronchial asthma has been caused by allergic symptoms. By enabling reliable diagnosis of bronchial asthma caused by allergies, accurate therapy can be selected at an early stage.

IL-4 and IL-13 are important factors for enhancing allergic reactions. Therefore, genes that increase their expression accompanying stimulation by these factors are considered to accomplish important roles in pathological formation of allergic symptoms. Furthermore, any of the indicator genes provided by this invention showed clear enhancement of expression in a plurality of respiratory tract epithelial cells. Studies focusing on the variance in gene expression level accompanying stimulation by IL-4 or IL-13 are not novel. However, the genes provided by this invention are all allergy related genes found by stringent criteria such as those described herein. Therefore, the indicator genes of this invention can be considered to be genes accomplishing an important role in allergic reactions compared to known allergy related genes obtained by similar approaches. The reason is that the indicator genes of this invention always respond sensitively to IL-4 or IL-13 stimulation in different cells. This supports the fact that the existence of indicator genes of this invention is indispensable to allergic reactions.

Thus, since excess expression of any indicator genes of this invention is linked to pathology, not only does suppression of these genes become the target of therapeutic strategy for allergic diseases, but also utility can be expected for these genes as novel clinical diagnostic indicators for monitoring in such novel treatment.

The expression level of indicator genes provided by this invention can be easily known, regardless of the type of allergen. Therefore, the pathology of an allergic reaction can be understood overall.

Additionally, the method of testing for allergies of this invention has low invasiveness towards patients since analysis of expression level can be carried out using a biological sample as the sample. Furthermore, regarding gene expression analysis, highly sensitive measurements are possible using small amounts of samples. Year after year, high throughput methods and decrease in cost are progressing in gene analysis technology. Therefore, in the near future, the method of testing for allergies of this invention is expected to become an important bed-side diagnostic method. In this sense, diagnostic value of these pathology related genes is high.

We claim:

1. A method of testing for an allergic disease, the method comprising the steps of:
a) measuring the expression level of one or more genes selected from the group consisting of carboxypeptidase M, cathepsin C, endothelin-A receptor, osteoblast specific factor 2, DD96, and CYP1B1 in a biological sample from a subject, and
b) comparing the expression level measured in (a) with that in a biological sample from a normal healthy subject.

2. The method of claim 1, wherein the allergic disease is bronchial asthma.

3. The method of claim 1, wherein the expression level is measured by PCR of the cDNA for the one or more genes.

4. The method of claim 1, wherein the expression level is measured by detecting the protein encoded by the one or more genes.

5. A reagent for testing for an allergic disease, said reagent comprising an oligonucleotide that is at least 15 nucleotides long and that has a nucleotide sequence complementary to a polynucleotide having a nucleotide sequence of one or more genes selected from the group consisting of carboxypeptidase M, cathepsin C, endothelin-A receptor, osteoblast specific factor 2, DD96, and CYP1B1 or to a complementary strand of the polynucleotide.

6. A reagent for testing for an allergic disease, said reagent comprising an antibody that recognizes a peptide having an amino acid sequence of one or more proteins selected from the group consisting of carboxypeptidase M, cathepsin C, endothelin-A receptor, osteoblast specific factor 2, DD96, and CYP1B1.

7. A method of screening for a therapeutic agent for an allergic disease, the method comprising the steps of:
(1) contacting a candidate compound with a cell that expresses one or more genes selected from the group consisting of carboxypeptidase M, cathepsin C, endothelin-A receptor, osteoblast specific factor 2, DD96, and CYP1B1, and/or one or more genes functionally equivalent thereto,
(2) measuring the expression level of the one or more genes, and
(3) selecting a compound that lowers the expression level, compared to a control.

8. The method of claim 7, wherein the cell is a respiratory tract epithelial cell line.

9. A method of screening for a therapeutic agent for an allergic disease, the method comprising the steps of:
(1) administering a candidate compound to a test animal, (2) measuring, in a biological sample from the test animal, the expression level of one or more genes selected from the group consisting of carboxypeptidase M, cathepsin C, endothelin-A receptor, osteoblast specific factor 2, DD96, and CYP1B1, and/or one or more genes functionally equivalent thereto, and
(3) selecting a compound that lowers the expression level of the one or more genes, compared to a control.

10. A method of screening for a therapeutic agent for an allergic disease, the method comprising the steps of:
(1) contacting a candidate substance with a cell transfected with a vector having a transcription regulatory region of one or more genes selected from the group consisting of carboxypeptidase M, cathepsin C, endothelin-A receptor, osteoblast specific factor 2, DD96, and CYP1B1, and/or one or more genes functionally equivalent thereto, and a reporter gene that is expressed under the control of the transcription regulatory region,
(2) measuring activity of the reporter gene, and
(3) selecting a compound that lowers the expression level of the reporter gene, compared to a control.

11. A method of screening for a therapeutic agent for an allergic disease, the method comprising the steps of:
(1) contacting a candidate substance with one or more proteins selected from the group consisting of carboxypeptidase M, cathepsin C, endothelin-A receptor, osteoblast specific factor 2, DD96, and CYP1B1, and/or one or more proteins functionally equivalent thereto,
(2) measuring activity of the one or more proteins, and
(3) selecting a compound that lowers the activity, compared to a control.

12. A therapeutic agent for an allergic disease, said agent comprising, as an active ingredient, a compound obtained by the method of any one of claims 7, 9, 10, and 11.

13. A therapeutic agent for an allergic disease, said agent comprising, as a major component, an antisense DNA against one or more genes selected from the group consisting of carboxypeptidase M, cathepsin C, endothelin-A receptor, osteoblast specific factor 2, DD96, and CYP1B1 or against a portion thereof.

14. A therapeutic agent for an allergic disease, said agent comprising, as a major component, an antibody that binds to one or more proteins selected from the group consisting of carboxypeptidase M, cathepsin C, endothelin-A receptor, osteoblast specific factor 2, DD96, and CYP1B1.

15. Use of a transgenic non-human vertebrate in which the expression level of one or more genes selected from the group consisting of carboxypeptidase M, cathepsin C, endothelin-A receptor, osteoblast specific factor 2, DD96, and CYP1B1, and/or one or more genes functionally equivalent thereto is elevated in respiratory tract epithelial cells, as an animal model for an allergic disease.

16. A kit for screening for a candidate compound for a therapeutic agent for an allergic disease, the kit comprising
a polynucleotide that is at least 15 nucleotides long and that hybridizes to a nucleotide sequence of one or more genes selected from the group consisting of carboxypeptidase M, cathepsin C, endothelin-A receptor, osteoblast specific factor 2, DD96(MAP17), and CYP1B1 or to a complementary sequence thereof, and
a cell that expresses a gene comprising a nucleotide sequence of one or more genes selected from the group consisting of carboxypeptidase M, cathepsin C, endothelin-A receptor, osteoblast specific factor 2, DD96(MAP17), and CYP1B1.

17. A kit for screening for a candidate compound for a therapeutic agent for an allergic disease, the kit comprising
an antibody that recognizes a peptide having an amino acid sequence of one or more proteins selected from the group consisting of carboxypeptidase M, cathepsin C, endothelin-A receptor, osteoblast specific factor 2, DD96(MAP17), and CYP1B1, and
a cell that expresses a gene comprising a nucleotide sequence of one or more genes selected from the group consisting of carboxypeptidase M, cathepsin C, endothelin-A receptor, osteoblast specific factor 2, DD96(MAP17), and CYP1B1.


SEQUENCE LISTING (37 sequences)

SEQ ID NO: 1 - DNA, 63 bp, Artificial Sequence
  Description of Artificial Sequencean artificially synthesized T7-d(T)24 primer sequence
ggccagtgaattgtaatacgactcactatagggaggcggtttttttttttttttttttttttt

SEQ ID NO: 2 - DNA, 23 bp, Artificial Sequence
  Description of Artificial Sequencean artificially synthesized primer sequence
acagagacgtttgtcctctctgc

SEQ ID NO: 3 - DNA, 21 bp, Artificial Sequence
  Description of Artificial Sequencean artificially synthesized primer sequence
atgccccagttgcttgaacac

SEQ ID NO: 4 - DNA, 27 bp, Artificial Sequence
  Description of Artificial Sequencean artificially synthesized TaqMan probe sequence
ccctcgtggccagttacccatttgata

SEQ ID NO: 5 - DNA, 22 bp, Artificial Sequence
  Description of Artificial Sequencean artificially synthesized primer sequence
tctcagaccccaatcctaagcc

SEQ ID NO: 6 - DNA, 22 bp, Artificial Sequence
  Description of Artificial Sequencean artificially synthesized primer sequence
ctgcaataaggtatgggaagcc

SEQ ID NO: 7 - DNA, 30 bp, Artificial Sequence
  Description of Artificial Sequencean artificially synthesized TaqMan probe sequence
tcttgtagccagtatgctcaaggctgtgaa

SEQ ID NO: 8 - DNA, 23 bp, Artificial Sequence
  Description of Artificial Sequencean artificially synthesized primer sequence
acctctgcgctcttagtgttgac

SEQ ID NO: 9 - DNA, 22 bp, Artificial Sequence
  Description of Artificial Sequencean artificially synthesized primer sequence
gcagttaccaaaggaatcccaa

SEQ ID NO: 10 - DNA, 30 bp, Artificial Sequence
  Description of Artificial Sequencean artificially synthesized TaqMan probe sequence
tacagagcagttgcctcctggagtcgtgtt

SEQ ID NO: 11 - DNA, 21 bp, Artificial Sequence
  Description of Artificial Sequencean artificially synthesized primer sequence
agcaaaccaccttcacggatc

SEQ ID NO: 12 - DNA, 23 bp, Artificial Sequence
  Description of Artificial Sequencean artificially synthesized primer sequence
ggtgccagcaaagtgtattctcc

SEQ ID NO: 13 - DNA, 28 bp, Artificial Sequence
  Description of Artificial Sequencean artificially synthesized TaqMan probe sequence
aattaggcttggcatctgctctgaggcc

SEQ ID NO: 14 - DNA, 23 bp, Artificial Sequence
  Description of Artificial Sequencean artificially synthesized primer sequence
gcctttgcagtcaaccacttctg

SEQ ID NO: 15 - DNA, 22 bp, Artificial Sequence
  Description of Artificial Sequencean artificially synthesized primer sequence
tctgttcccaccaggactccat

SEQ ID NO: 16 - DNA, 26 bp, Artificial Sequence
  Description of Artificial Sequencean artificially synthesized TaqMan probe sequence
atgatcctgaccgtcggaaacaaggc

SEQ ID NO: 17 - DNA, 20 bp, Artificial Sequence
  Description of Artificial Sequencean artificially synthesized primer sequence
ttatgaagccatgcgcttct

SEQ ID NO: 18 - DNA, 21 bp, Artificial Sequence
  Description of Artificial Sequencean artificially synthesized primer sequence
aagacagaggtgttggcagtg

SEQ ID NO: 19 - DNA, 29 bp, Artificial Sequence
  Description of Artificial Sequencean artificially synthesized TaqMan probe sequence
cagctttgtgcctgtcactattcctcatg

SEQ ID NO: 20 - DNA, 25 bp, Artificial Sequence
  Description of Artificial Sequencean artificially synthesized primer sequence
tcacccacactgtgcccatctacga

SEQ ID NO: 21 - DNA, 25 bp, Artificial Sequence
  Description of Artificial Sequencean artificially synthesized primer sequence
cagcggaaccgctcattgccaatgg

SEQ ID NO: 22 - DNA, 26 bp, Artificial Sequence
  Description of Artificial Sequencean artificially synthesized TaqMan probe sequence
atgccctcccccatgccatcctgcgt

SEQ ID NO: 23 - DNA, 18 bp, Artificial Sequence
  Description of Artificial Sequencean artificially synthesized primer sequence
gaaggtgaaggtcggagt

SEQ ID NO: 24 - DNA, 20 bp, Artificial Sequence
  Description of Artificial Sequencean artificially synthesized primer sequence
gaagatggtgatgggatttc

SEQ ID NO: 25 - DNA, 20 bp, Artificial Sequence
  Description of Artificial Sequencean artificially synthesized TaqMan probe sequence
caagcttcccgttctcagcc

SEQ ID NO: 26 - DNA, 2085 bp, Homo sapiens
gcatttcttccttctgcgtatgggacaggaccctttctggaatgggggtcttatgacctacaatcaaacaagaacatggacttcccgtgcctctggctagggctgttgctgcctttggtagctgcgctggatttcaactaccaccgccaggaagggatggaagcgtttttgaagactgttgcccaaaactacagttctgtcactcacttacacagtattgggaaatctgtgaaaggtagaaacctgtgggttcttgttgtggggcggtttccaaaggaacacagaattgggattccagagttcaaatacgtggcaaatatgcatggagatgagactgttgggcgggagctgctgctccatctgattgactatctcgtaaccagtgatggcaaagaccctgaaatcacaaatctgatcaatagtacccggatacacatcatgccttccatgaacccagatggatttgaagccgtcaaaaagcctgactgttactacagcatcggaagggaaaattataaccagtatgacttgaatcgaaatttccccgatgcttttgaatataataatgtctcaaggcagcctgaaactgtggcagtcatgaagtggctgaaaacagagacgtttgtcctctctgcaaacctccatggtggtgccctcgtggccagttacccatttgataatggtgttcaagcaactggggcattatactcccgaagcttaacgcctgatgatgatgtttttcaatatcttgcacatacctatgcttcaagaaatcccaacatgaagaaaggagacgagtgtaaaaacaaaatgaactttcctaatggtgttacaaatggatactcttggtatccactccaaggtggaatgcaagattacaactacatctgggcccagtgttttgaaattacgttggagctgtcatgctgtaaatatcctcgtgaggagaagcttccatccttttggaataataacaaagcctcattaattgaatatataaagcaggtgcacctaggtgtaaagggtcaagtttttgatcagaatggaaatccattacccaatgtaattgtggaagtccaagacagaaaacatatctgcccctatagaaccaacaaatatggagagtattatctccttctcttgcctgggtcttatattataaatgttacagtccctggacatgatccacacatcacaaaggtgattattccggagaaatcccagaacttcagtgctcttaaaaaggatattctacttccattccaagggcaattggattctatcccagtatcaaatccttcatgcccaatgattcctctatacagaaatttgccagaccactcagctgcaacaaagcctagtttgttcttatttttagtgagtcttttgcacatattcttcaaataaagtaaaatgtgaaactcaacccacatcaccacctggaatcagggattgctcactccaggttactgcaaccctaactcactctagtgggaccttgactggagaaactccacgatcttcctgaagaagagaaatggatgtttccaaattccacaataagcaatatgtggtgataatgaaaagaatgattcagtcttgacggtgaatggaagacacttacctaacaagtactgctcatttacactcaaattaatcttgaagtagtcttaaaatgtgtaagaagttaaaacttgagaagcaaaaaatgcctgcaaaaagaagatcattttgtatacagagaaccggatgaatataagcaatgaagatgaacatttattgatcttctacatacaagacttcaccataaggccaggagcagtgctcacgccttgtaatcccagcactttgggaggccaaggtgggcggatcaccttgaggtcaggagttcaagaccagcctgaccaacatggtgaaaccctgtctctactaaatattagcggggtgtggtggcgggcacctgtagtcgcagcctttcgggaggctgagacaggagaatcgcttgaaccctagaggcggagtttgcagtgagccgagatagtgccattgtactccagcttgggcaacagagtaagactctgtctc

SEQ ID NO: 27 - protein, 443 aa, Homo sapiens
MDFPCLWLGLLLPLVAALDFNYHRQEGMEAFLKTVAQNYSSVTHLHSIGKSVKGRNLWVLVVGRFPKEHRIGIPEFKYVANMHGDETVGRELLLHLIDYLVTSDGKDPEITNLINSTRIHIMPSMNPDGFEAVKKPDCYYSIGRENYNQYDLNRNFPDAFEYNNVSRQPETVAVMKWLKTETFVLSANLHGGALVASYPFDNGVQATGALYSRSLTPDDDVFQYLAHTYASRNPNMKKGDECKNKMNFPNGVTNGYSWYPLQGGMQDYNYIWAQCFEITLELSCCKYPREEKLPSFWNNNKASLIEYIKQVHLGVKGQVFDQNGNPLPNVIVEVQDRKHICPYRTNKYGEYYLLLLPGSYIINVTVPGHDPHITKVIIPEKSQNFSALKKDILLPFQGQLDSIPVSNPSCPMIPLYRNLPDHSAATKPSLFLFLVSLLHIFFK

SEQ ID NO: 28 - DNA, 1838 bp, Homo sapiens
aattcttcacctcttttctcagctccctgcagcatgggtgctgggccctccttgctgctcgccgccctcctgctgcttctctccggcgacggcgccgtgcgctgcgacacacctgccaactgcacctatcttgacctgctgggcacctgggtcttccaggtgggctccagcggttcccagcgcgatgtcaactgctcggttatgggaccacaagaaaaaaaagtagtggtgtaccttcagaagctggatacagcatatgatgaccttggcaattctggccatttcaccatcatttacaaccaaggctttgagattgtgttgaatgactacaagtggtttgccttttttaagtataaagaagagggcagcaaggtgaccacttactgcaacgagacaatgactgggtgggtgcatgatgtgttgggccggaactgggcttgtttcaccggaaagaaggtgggaactgcctctgagaatgtgtatgtcaacacagcacaccttaagaattctcaggaaaagtattctaataggctctacaagtatgatcacaactttgtgaaagctatcaatgccattcagaagtcttggactgcaactacatacatggaatatgagactcttaccctgggagatatgattaggagaagtggtggccacagtcgaaaaatcccaaggcccaaacctgcaccactgactgctgaaatacagcaaaagattttgcatttgccaacatcttgggactggagaaatgttcatggtatcaattttgtcagtcctgttcgaaaccaagcatcctgtggcagctgctactcatttgcttctatgggtatgctagaagcgagaatccgtatactaaccaacaattctcagaccccaatcctaagccctcaggaggttgtgtcttgtagccagtatgctcaaggctgtgaaggcggcttcccataccttattgcaggaaagtacgcccaagattttgggctggtggaagaagcttgcttcccctacacaggcactgattctccatgcaaaatgaaggaagactgctttcgttattactcctctgagtaccactatgtaggaggtttctatggaggctgcaatgaagccctgatgaagcttgagttggtccatcatgggcccatggcagttgcttttgaagtatatgatgacttcctccactacaaaaaggggatctaccaccacactggtctaagagaccctttcaacccctttgagctgactaatcatgctgttctgcttgtgggctatggcactgactcagcctctgggatggattactggattgttaaaaacagctggggcaccggctggggtgagaatggctacttccggatccgcagaggaactgatgagtgtgcaattgagagcatagcagtggcagccacaccaattcctaaattgtagggtatgccttccagtatttcataatgatctgcatcagttgtaaaggggaattggtatattcacagactgtagactttcagcagcaatctcagaagcttacaaatagatttccatgaagatatttgtcttcagaattaaaactgcccttaattttaatatacctttcaatcggccactggccatttttttctaagtattcaattaagtgggaattttctggaagatggtcagctatgaagtaatagagtttgcttaatcatttgtaattcaaacatgctatattttttaaaatcaatgtgaaaacatagacttatttttaaattgtaccaatcacaagaaaataatggcaataattatcaaaacttttaaaatagatgctcatatttttaaaataaagttttaaaaataactgc

SEQ ID NO: 29 - protein, 463 aa, Homo sapiens
MGAGPSLLLAALLLLLSGDGAVRCDTPANCTYLDLLGTWVFQVGSSGSQRDVNCSVMGPQEKKVVVYLQKLDTAYDDLGNSGHFTIIYNQGFEIVLNDYKWFAFFKYKEEGSKVTTYCNETMTGWVHDVLGRNWACFTGKKVGTASENVYVNTAHLKNSQEKYSNRLYKYDHNFVKAINAIQKSWTATTYMEYETLTLGDMIRRSGGHSRKIPRPKPAPLTAEIQQKILHLPTSWDWRNVHGINFVSPVRNQASCGSCYSFASMGMLEARIRILTNNSQTPILSPQEVVSCSQYAQGCEGGFPYLIAGKYAQDFGLVEEACFPYTGTDSPCKMKEDCFRYYSSEYHYVGGFYGGCNEALMKLELVHHGPMAVAFEVYDDFLHYKKGIYHHTGLRDPFNPFELTNHAVLLVGYGTDSASGMDYWIVKNSWGTGWGENGYFRIRRGTDECAIESIAVAATPIPKL

SEQ ID NO: 30 - DNA, 2595 bp, Homo sapiens
gtctgttccttcccccagtcatgcctctgctgctgctgttaccagtccaaaagtctgatgacctcggtccccatgaacggaacaagcatccagtggaagaaccacgatcaaaacaaccacaacacagaccggagcagccataaggacagcatgaactgaccacccttagaagcactcctcggtactcccataatcctctcggagaaaaaaatcacaaggcaactgtgactccgggaatctcttctctgatccttcttccttaattcactcccacacccaagaagaaatgctttccaaaaccgcaaggtagactggtttatccacccacaacatctacgaatcgtacttctttaattgatctaatttacatattctgcgtgttgtattcagcactaaaaaatggtgggagctgggggagaatgaagactgttaaatgaaaccagaaggatatttactacttttgcatgaaaatagagctttcaagtacatggctagcttttatggcagttctggtgaatgttcaatgggaactggtcaccatgaaactttagagattaacgacaagattttctactttttttaagtgattttttgtccttcagccaaacacaatatgggctcaggtcacttttatttgaaatgtcatttggtgccagtattttttaactgcataatagcctaacatgattatttgaacttatttacacatagtttgaaaaaaaaaagacaaaaatagtattcaggtgagcaattagattagtattttccacgtcactatttatttttttaaaacacaaattctaaagctacaacaaatactacaggcccttaaagcacagtctgatgacacatttggcagtttaatagatgttactcaaagaattttttaagaactgtattttattttttaaatggtgttttattacaagggaccttgaacatgttttgtatgttaaattcaaaagtaatgcttcaatcagatagttctttttcacaagttcaatctgtttttcatgtaaattttgtatgaaaaatcaatgtcaagtaccaaaatgttaatgtatgtgtcatttaactctgcctgagactttcagtgcactgtatatagaagtctaaaacacacctaagagaaaaagatcgaatttttcagatgattcggaaattttcattcaggtatttgtaatagtgacatatatatgtatatacatatcacctcctattctcttaatttttgttaaaatgttaactggcagtaagtcttttttgatcattcccttttccatataggaaacataattttgaagtggccagatgagtttatcatgtcagtgaaaaataattacccacaaatgccaccagtaacttaacgattcttcacttcttggggttttcagtatgaacctaactccccaccccaacatctccctcccacattgtcaccatttcaaagggcccacagtgacttttgctgggcattttcccagatgtttacagactgtgagtacagcagaaaatcttttactagtgtgtgtgtgtatatatataaacaattgtaaatttcttttagcccatttttctagactgtctctgtggaatatatttgtgtgtgtgatatatgcatgtgtgtgatggtatgtatggatttaatctaatctaataattgtgccccgcagttgtgccaaagtgcatagtctgagctaaaatctaggtgattgttcatcatgacaacctgcctcagtccattttaacctgtagcaaccttctgcattcataaatcttgtaatcatgttaccattacaaatgggatataagaggcagcgtgaaagcagatgagctgtggactagcaatatagggttttgtttggttggttggtttgataaagcagtatttggggtcatattgtttcctgtgctggagcaaaagtcattacactttgaagtattatattgttcttatcctcaattcaatgtggtgatgaaattgccaggttgtctgatatttctttcagacttcgccagacagattgctgataataaattaggtaagataatttgttgggccatattttaggacaggtaaaataacatcaggttccagttgcttgaattgcaaggctaagaagtactgcccttttgtgtgttagcagtcaaatctattattccactggcgcatcatatgcagtgatatatgcctataatataagccataggttcacaccattttgtttagacaattgtctttttttcaagatgctttgtttctttcatatgaaaaaaatgcattttataaattcagaaagtcatagatttctgaaggcgtcaacgtgcattttatttatggactggtaagtaactgtggtttactagcaggaatatttccaatttctacctttactacatcttttcaacaagtaactttgtagaaatgagccagaagccaaggccctgagttggcagtggcccataagtgtaaaataaaagtttacagaaaccttgcaagtgtctcttcatttttatgtagttttccatagaaaatgtttgttacataatgcctgttgcaaacctctccttagtaatatcctggaaagcagttta

SEQ ID NO: 31 - protein, 427 aa, Homo sapiens
METLCLRASFWLALVGCVISDNPERYSTNLSNHVDDFTTFRGTELSFLVTTHQPTNLVLPSNGSMHNYCPQQTKITSAFKYINTVISCTIFIVGMVGNATLLRIIYQNKCMRNGPNALIASLALGDLIYVVIDLPINVFKLLAGRWPFDHNDFGVFLCKLFPFLQKSSVGITVLNLCALSVDRYRAVASWSRVQGIGIPLVTAIEIVSIWILSFILAIPEAIGFVMVPFEYRGEQHKTCMLNATSKFMEFYQDVKDWWLFGFYFCMPLVCTAIFYTLMTCEMLNRRNGSLRIALSEHLKQRREVAKTVFCLVVIFALCWFPLHLSRILKKTVYNEMDKNRCELLSFLLLMDYIGINLATMNSCINPIALYFVSKKFKNCFQSCLCCCCYQSKSLMTSVPMNGTSIQWKNHDQNNHNTDRSSHKDSMN

SEQ ID NO: 32 - DNA, 3213 bp, Homo sapiens
agagactcaagatgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgttttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggttcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggttcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatacaactaaaattataaccaaagttgtggaaccaaaaattaaagtgattgaaggcagtcttcagcctattatcaaaactgaaggacccacactaacaaaagtcaaaattgaaggtgaacctgaattcagactgattaaagaaggtgaaacaataactgaagtgatccatggagagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggttctagaagacgattaagggaaggtcgttctcagtgaaaatccaaaaaccagaaaaaaatgtttatacaaccctaagtcaataacctgaccttagaaaattgtgagagccaagttgacttcaggaactgaaacatcagcacaaagaagcaatcatcaaataattctgaacacaaatttaatatttttttttctgaatgagaaacatgagggaaattgtggagttagcctcctgtggtaaaggaattgaagaaaatataacaccttacaccctttttcatcttgacattaaaagttctggctaactttggaatccattagagaaaaatccttgtcaccagattcattacaattcaaatcgaagagttgtgaactgttatcccattgaaaagaccgagccttgtatgtatgttatggatacataaaatgcacgcaagccattatctctccatgggaagctaagttataaaaataggtgcttggtgtacaaaactttttatatcaaaaggctttgcacatttctatatgagtgggtttactggtaaattatgttattttttacaactaattttgtactctcagaatgtttgtcatatgcttcttgcaatgcatattttttaatctcaaacgtttcaataaaaccatttttcagatataaagagaattacttcaaattgagtaattcagaaaaactcaagatttaagttaaaaagtggtttggacttgggaa

SEQ ID NO: 33 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMVQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 34 - DNA, 927 bp, Homo sapiens
ggaagtttaggttaactgtcttaaatttccaaagctgtaatcattattttcattctcaaagtgatggccttgtgttttgctcctctcctccagggccagactgagcccaggttgatttcaggcggacaccaatagactccacagcagctccaggagcccagacaccggcggccagaagcaaggctaggagctgctgcagccatgtcggccctcagcctcctcattctgggcctgctcacggcagtgccacctgccagctgtcagcaaggcctggggaaccttcagccctggatgcagggccttatcgcggtggccgtgttcctggtcctcgttgcaatcgcctttgcagtcaaccacttctggtgccaggaggagccggagcctgcacacatgatcctgaccgtcggaaacaaggcagatggagtcctggtgggaacagatggaaggtactcttcgatggcggccagtttcaggtccagtgagcatgagaatgcctatgagaatgtgcccgaggaggaaggcaaggtccgcagcaccccgatgtaaccttctctgtggctccaaccccaagactcccaggcacatgggatggatgtccagtgctaccacccaagccccctccttctttgtgtggaatctgcaatagtgggctgactccctccagccccatgccggccctacccgcccttgaagtatagccagccaaggttggagctcagaccgtgtctaggttggggctcggctgtggccctggggtctcctgctcagctcagaagagccttctggagaggacagtcagctgagcacctcccatcctgctcacacgtccttccccataactatggaaatggccctaatttctgtgaaataaagactttttgtatttctggggctgaggctcagcaacagcccctcaggcttccaaaa

SEQ ID NO: 35 - protein, 114 aa, Homo sapiens
MSALSLLILGLLTAVPPASCQQGLGNLQPWMQGLIAVAVFLVLVAIAFAVNHFWCQEEPEPAHMILTVGNKADGVLVGTDGRYSSMAASFRSSEHENAYENVPEEEGKVRSTPM

SEQ ID NO: 36 - DNA, 5102 bp, Homo sapiens
gcttctgcgactccagttgtgagagccgcaagggcatgggaattgacgccactcaccgacccccagtctcaatctcaacgctgtgaggaaacctcgactttgccaggtccccaagggcagcggggctcggcgagcgaggcacccttctccgtccccatcccaatccaagcgctcctggcactgacgacgccaagagactcgagtgggagttaaagcttccagtgagggcagcaggtgtccaggccgggcctgcgggttcctgttgacgtcttgccctaggcaaaggtcccagttccttctcggagccggctgtcccgcgccactggaaaccgcacctccccgcagcatgggcaccagcctcagcccgaacgacccttggccgctaaacccgctgtccatccagcagaccacgctcctgctactcctgtcggtgctggccactgtgcatgtgggccagcggctgctgaggcaacggaggcggcagctccggtccgcgcccccgggcccgtttgcgtggccactgatcggaaacgcggcggcggtgggccaggcggctcacctctcgttcgctcgcctggcgcggcgctacggcgacgttttccagatccgcctgggcagctgccccatagtggtgctgaatggcgagcgcgccatccaccaggccctggtgcagcagggctcggccttcgccgaccggccggccttcgcctccttccgtgtggtgtccggcggccgcagcatggctttcggccactactcggagcactggaaggtgcagcggcgcgcagcccacagcatgatgcgcaacttcttcacgcgccagccgcgcagccgccaagtcctcgagggccacgtgctgagcgaggcgcgcgagctggtggcgctgctggtgcgcggcagcgcggacggcgccttcctcgacccgaggccgctgaccgtcgtggccgtggccaacgtcatgagtgccgtgtgtttcggctgccgctacagccacgacgaccccgagttccgtgagctgctcagccacaacgaagagttcgggcgcacggtgggcgcgggcagcctggtggacgtgatgccctggctgcagtacttccccaacccggtgcgcaccgttttccgcgaattcgagcagctcaaccgcaacttcagcaacttcatcctggacaagttcttgaggcactgcgaaagccttcggcccggggccgccccccgcgacatgatggacgcctttatcctctctgcggaaaagaaggcggccggggactcgcacggtggtggcgcgcggctggatttggagaacgtaccggccactatcactgacatcttcggcgccagccaggacaccctgtccaccgcgctgcagtggctgctcctcctcttcaccaggtatcctgatgtgcagactcgagtgcaggcagaattggatcaggtcgtggggagggaccgtctgccttgtatgggtgaccagcccaacctgccctatgtcctggccttcctttatgaagccatgcgcttctccagctttgtgcctgtcactattcctcatgccaccactgccaacacctctgtcttgggctaccacattcccaaggacactgtggtttttgtcaaccagtggtctgtgaatcatgacccagtgaagtggcctaacccggagaactttgatccagctcgattcttggacaaggatggcctcatcaacaaggacctgaccagcagagtgatgattttttcagtgggcaaaaggcggtgcattggcgaagaactttctaagatgcagctttttctcttcatctccatcctggctcaccagtgcgatttcagggccaacccaaatgagcctgcgaaaatgaatttcagttatggtctaaccattaaacccaagtcatttaaagtcaatgtcactctcagagagtccatggagctccttgatagtgctgtccaaaatttacaagccaaggaaacttgccaataagaagcaagaggcaagctgaaattttagaaatattcacatcttcggagatgaggagtaaaattcagtttttttccagttcctcttttgtgctgcttctcaattagcgtttaaggtgagcataaatcaactgtccatcaggtgaggtgtgctccatacccagcggttcttcatgagtagtgggctatgcaggagcttctgggagatttttttgagtcaaagacttaaagggcccaatgaattattatatacatactgcatcttggttatttctgaaggtagcattctttggagttaaaatgcacatatagacacatacacccaaacacttacaccaaactactgaatgaagaagtattttggtaaccaggccatttttggtgggaatccaagattggtctcccatatgcagaaatagacaaaaagtatattaaacaaagtttcagagtatattgttgaagagacagagacaagtaatttcagtgtaaagtgtgtgattgaaggtgataagggaaaagataaagaccagaaattcccttttcaccttttcaggaaaataacttagactctagtatttatgggtggatttatccttttgccttctggtatacttccttacttttaaggataaatcataaagtcagttgctcaaaaagaaatcaatagttgaattagtgagtatagtggggttccatgagttatcatgaattttaaagtatgcattattaaattgtaaaactccaaggtgatgttgtacctcttttgcttgccaaagtacagaatttgaattatcagcaaagaaaaaaaaaaaagccagccaagctttaaattatgtgaccataatgtactgatttcagtaagtctcataggttaaaaaaaaaagtcaccaaatagtgtgaaatatattacttaactgtccgtaagcagtatattagtattatcttgttcaggaaaaggttgaataatatatgccttgtgtaatattgaaaattgaaaagtacaactaacgcaaccaagtgtgctaaaaatgagcttgattaaatcaaccacctatttttgacatggaaatgaagcagggtttcttttcttcactcaaattttggcgaatctcaaaattagatcctaagatgtgttcttatttttataacatctttattgaaattctatttataatacagaatcttgttttgaaaataacctaattaatatattaaaattccaaattcatggcatgcttaaattttaactaaattttaaagccattctgattattgagttccagttgaagttagtggaaatctgaacattctcctgtggaaggcagagaaatctaagctgtgtctgcccaatgaataatggaaaatgccatgaattacctggatgttctttttacgaggtgacaagagttggggacagaactcccattacaactgaccaagtttctcttctagatgattttttgaaagttaacattaatgcctgctttttggaaagtcagaatcagaagatagtcttggaagctgtttggaaaagacagtggagatgaggtcagttgtgttttttaagatggcaattactttggtagctgggaaagcataaagctcaaatgaaatgtatgcattcacatttagaaaagtgaattgaagtttcaagttttaaagttcattgcaattaaacttccaaagaaagttctacagtgtcctaagtgctaagtgcttattacattttattaagctttttggaatctttgtaccaaaattttaaaaaagggagtttttgatagttgtgtgtatgtgtgtgtggggtggggggatggtaagagaaaagagagaaacactgaaaagaaggaaagatggttaaacattttcccactcattctgaattaattaatttggagcacaaaattcaaagcatggacatttagaagaaagatgtttggcgtagcagagttaaatctcaaataggctattaaaaaagtctacaacatagcagatctgttttgtggtttggaatattaaaaaacttcatgtaattttattttaaaatttcatagctgtacttcttgaatataaaaaatcatgccagtatttttaaaggcattagagtcaactacacaaagcaggcttgcccagtacatttaaattttttggcacttgccattccaaaatattatgccccaccaaggctgagacagtgaatttgggctgctgtagcctatttttttagattgagaaatgtgtagctgcaaaaataatcatgaaccaatctggatgcctcattatgtcaaccaggtccagatgtgctataatctgtttttacgtatgtaggcccagtcgtcatcagatgcttgcggcaaaagaaagctgtgtttatatggaagaaagtaaggtgcttggagtttacctggcttatttaatatgcttataacctagttaaagaaaggaaaagaaaacaaaaaacgaatgaaaataactgaatttggaggctggagtaatcagattactgctttaatcagaaaccctcattgtgtttctaccggagagagaatgtatttgctgacaaccattaaagtcagaagttttactccaggttattgcaataaagtataatgtttattaaatgcttcatttgtatgtcaaagctttgactctataagcaaattgcttttttccaaaacaaaaagatgtctcaggtttgttttgtgaattttctaaaagctttcatgtcccagaacttagcctttacctgtgaagtgttactacagccttaatattttcctagtagatctatattagatcaaatagttgcatagcagtatatgttaatttgtgtgtttttagctgtgacacaactgtgtgattaaaaggtatactttagtagacatttataactcaaggataccttcttatttaatcttttcttatttttgtactttatcatgaatgcttttagtgtgtgcataatagctacagtgcatagttgtagacaaagtacattctggggaaacaacatttatatgtagcctttactgtttgatataccaaattaaaaaaaaattgtatctcattacttatactgggacaccattaccaaaataataaaaatcactttcataatcttgaaaaaa

SEQ ID NO: 37 - protein, 543 aa, Homo sapiens
MGTSLSPNDPWPLNPLSIQQTTLLLLLSVLATVHVGQRLLRQRRRQLRSAPPGPFAWPLIGNAAAVGQAAHLSFARLARRYGDVFQIRLGSCPIVVLNGERAIHQALVQQGSAFADRPAFASFRVVSGGRSMAFGHYSEHWKVQRRAAHSMMRNFFTRQPRSRQVLEGHVLSEARELVALLVRGSADGAFLDPRPLTVVAVANVMSAVCFGCRYSHDDPEFRELLSHNEEFGRTVGAGSLVDVMPWLQYFPNPVRTVFREFEQLNRNFSNFILDKFLRHCESLRPGAAPRDMMDAFILSAEKKAAGDSHGGGARLDLENVPATITDIFGASQDTLSTALQWLLLLFTRYPDVQTRVQAELDQVVGRDRLPCMGDQPNLPYVLAFLYEAMRFSSFVPVTIPHATTANTSVLGYHIPKDTVVFVNQWSVNHDPVKWPNPENFDPARFLDKDGLINKDLTSRVMIFSVGKRRCIGEELSKMQLFLFISILAHQCDFRANPNEPAKMNFSYGLTIKPKSFKVNVTLRESMELLDSAVQNLQAKETCQ